(19) United States
(12) Patent Application Publication
(10) Pub. No.: US 2004/0142851 A1
(43) Pub. Date: Jul. 22, 2004

(54) Hydrazinopeptoids and their uses for treating cancers

(76) Inventors: Yannick Bonnemains, Thorigne Fouillard (FR); Karine Bouget, St. Jouan des Guerets (FR); Michele Floc'h, Rennes (FR); Philippe Le Grel, Rennes (FR); Sandrine Aubin, Rennes (FR)

Correspondence Address: YOUNG & THOMPSON, 745 SOUTH 23RD STREET 2ND FLOOR, ARLINGTON, VA, 22202

(21) Appl. No.: 10/432,986

(22) PCT Filed: Aug. 27, 2002
(86) PCT No.: PCT/FR02/02935

(30) Foreign Application Priority Data
Aug. 27, 2001 (FR) 01/11120

(51) Int. Cl. A61K 31/275; A61K 38/00; A61K 31/22; A61K 31/16

(52) U.S. Cl. 514/2; 514/528; 514/551; 514/614

(57) ABSTRACT

Compounds of general formula (I), wherein: n represents an integer from 1 to 10; R₁ and R₆, independently of each other, represent a hydrogen atom, a group useful for protecting nitrogen atoms in peptide synthesis or a group of formula COR or —CH₂COR, wherein R represents a hydrogen atom, an alkyl group of 1 to 10 carbon atoms, a —COORₐ group wherein Rₐ represents H or an alkyl group, a primary —NH₂ amino group or a secondary or tertiary amine, an alkoxy group, a phenyl group or a pyridinium group; R₂, R₃, R₄ and R₅, independently of one another, represent a hydrogen atom, an alkyl group of 1 to 10 carbon atoms; Y represents CH₂ and Z represents CO, or Y represents CO and Z represents CH₂, for preparing a medicine for treating tumoral pathologies or neurodegenerative diseases such as Alzheimer's or Lehn's disease.

A subject of the present invention is the use of hydrazinopeptoid compounds in the treatment of tumours. A subject of the invention is also new hydrazinopeptoid compounds, as well as their synthesis processes.

The cell cycle of the majority of cells allows them to increase in size, double their quantity of DNA, and then to separate and divide their chromosomes in order to produce two daughter cells identical to each other and identical to the cell from which they are produced. The cell cycle is divided into two very distinct periods: the interphase during which the DNA replication and mitosis take place. The replication and mitosis phases are controlled by protein complexes regulated by their phosphorylation state and/or their degradation. A number of neurodegenerative and/or cancer pathologies, associated with the presence of incorrectly structured proteins (aberration in the secondary and tertiary structure of the molecule) or with the presence of proteins which are not degraded at a stage where it is essential that they are, are currently known.

The ubiquitin/proteasome system plays a major role in intracellular proteolysis, the degradation of a certain number of proteins associated with the correct development of the cell cycle. The inactivation of the proteasome by specific inhibitors of the active site makes it possible to understand the mechanism of protein degradation dysfunction and thus to envisage new classes of anti-tumour molecules.

It has been observed that peptide aldehyde inhibitors of calpain and proteasome such as N-acetyl-leucinyl-leucinyl-norleucinal (ALLN), benzyloxycarbonyl leucinyl-leucinyl-leucinal (MG132) and N-acetyl-leucinyl-valinyl-phenylalaninal (ALVP), but not N-acetyl-leucinyl-leucinyl-methioninal (ALLM), have a synergic action in the suppression of cell proliferation and the induction of apoptosis in three human tumour cell lines as well as in pulmonary adenocarcinomas, prostate carcinomas, and breast carcinomas (Cusak J C, Liu R, Houston M, Adendroth K, Elliot P J, Adams J and Baldwin A S Jr (2001) Cancer Res, 61, 3535-3540; Soligo D, Servida D, Fontanella E, Lamorte G, Caneva L, Fumiatti R, and Lambertenghi Deliliers G (2001) Br J Haematol, 113, 126-135; Sun J, Nam S, Lee C S, Li B, Coppola D, Hamilton A D, Dou Q P and Sebti S M (2001) Cancer Res, 61, 1280-1284).

The transformed peptides and in particular the pseudopeptides are arousing great interest as they are capable of behaving as more effective analogues than the peptides themselves, the therapeutic uses of which are however limited by considerable biodegradability, poor ability to clear the physiological barriers and by the lack of selectivity vis-à-vis the target. It is therefore necessary to design more active, more stable and more specific analogues. The pseudopeptides for which the chemical nature of the peptide skeleton and of the amide bond (CO—NH) is modified, make it possible to induce a much greater bioavailability than that of the mimicked peptides whilst retaining a good biological activity. This property of the pseudopeptides, such as the azapeptides and the peptoids, is linked in particular to the resistance induced vis-à-vis the peptidases, which very rapidly degrade any exogenous peptide by cutting the peptide skeleton at the level of the amide bonds, and the action of which is then slowed down by the modification of these bonds.

Precursor compounds in the field of hydrazinopeptoids, as well as their synthesis processes, are described in the article by Cheguillaume et al., J Org. Chem., 1999, 64, 2924-2927. However, this article does not describe any of the biological properties of these compounds.

Moreover, the article by Bouget et al. published in Peptides 2000, Jean Martinez and Jean-Alain Fehrentz (Eds.) EDK, Paris, France © 2001, pp 793-794, describes the effect of hydrazinopeptoid-type compounds in the inhibition of cell cycle progression. However, the results presented in this article can be linked to any non-specific mechanism of cancerous cells other than that involving the proteasome (such as the depolymerization of the microtubules leading to a disorganisation of the cytoskeleton and thus causing the cycle to stop), which would make it impossible to use the compounds described in this article in the treatment of cancers.

The present invention results from the demonstration by the Inventors of the fact that the hydrazinopeptoid compounds of formula (I), described hereafter, have a specific action on the cancerous cells by inducing the apoptosis of the latter according to a inhibition mechanism of the enzyme activities produced by the proteasome.

A subject of the invention is the use of compounds of the following general formula (I):

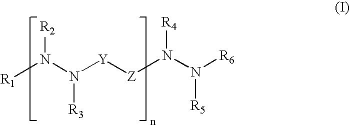

in which:

n represents an integer from 1 to 10, in particular n represents 1 or 2,

Y represents CH₂ and Z represents CO, or Y represents CO and Z represents CH₂,

R₁ and R₆, independently from one another, represent:

a hydrogen atom,

a group which can be used in the protection of the nitrogen atoms in peptide synthesis, such as the BOC, FMOC or Z group,

a group of formula —COR, or —CH₂COR in which R represents:

a hydrogen atom, except that, when R₁ is a hydrogen, this is presented in the form of a salt which is soluble in aqueous solvents, such as a trifluoroacetate salt,

an alkyl group with 1 to 10 carbon atoms, optionally substituted by one or more halogen atoms, such as the R groups representing —CF₃ or a —CH₂X group, X representing a halogen atom such as Cl or Br, or an abovementioned alkyl group substituted by a cyano group, such as the R group representing —CH₂—CN, or by a sulphurated group such as the R group representing —CH₂—SC₂H₅,

a —COORₐ group in which Rₐ represents H or an alkyl group, such as a methyl or ethyl group,

an —NH₂ primary amine or a secondary or tertiary amine group,

an alkoxy group, such as an —OMe methoxy, or —OEt ethoxy group,

a phenyl group,

a pyridinium group, such as the group of formula

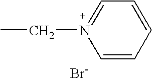

R₂, R₃, R₄ and R₅, independently from one another, representing:

a hydrogen atom,

an alkyl group with 1 to 10 carbon atoms, optionally substituted, in particular by one or more halogen atoms or by one or more amine or phenyl groups, such as the butyl, isobutyl, —(CH₂)₄NH₂, —CH₂Ph, —CH₂)₄NHBoc groups,

or R₁ in combination with R₂, or R₆ in combination with R₅, represents a group of formula

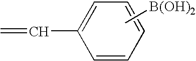

for the preparation of a medicament for the treatment of tumour pathologies or of neurodegenerative diseases such as Alzheimer's or Lehn's disease.

A more particular subject of the invention is the abovementioned use of compounds of general formula (I) in which:

R₁ represents a BOC, FMOC, Z group or H, except that, when R₁ represents H, this is presented in the form of a salt, such as a trifluoroacetate salt of formula CF₃CO₂⁻, H₃N⁺—,

R₂ represents H or an alkyl group with 1 to 10 carbon atoms, such as an isobutyl group,

R₃ represents H or an alkyl group with 1 to 10 carbon atoms, such as an isobutyl group,

one of R₄ or R₅ represents H, whilst the other represents an alkyl group as defined above, and R₆ represents a group of formula —COR or —CH₂COR as defined above,

or R₅ in combination with R₆ represents a group of formula

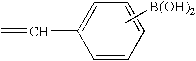

n represents 1 or 2,

Y and Z are as defined above.

The invention relates yet more particularly to the abovementioned use of compounds of general formula (I) in which R₅ represents H, and R₆ represents a —COR or —CH₂COR group in which R represents a —CH₂X group, X representing a halogen atom such as Cl or Br, or a pyridinium group.

A more particular subject of the invention is also the abovementioned use of compounds of general formula (I) in which R₅ represents H and R₆ represents a —COCH₂Br,—COCH₂Cl or

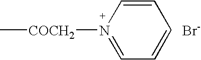

group

The invention relates yet more particularly to the abovementioned use of compounds of general formula (I) in which R₁ and R₂ represent H.

A more particular subject of the invention is the abovementioned use of the compounds of general formula (I) in which Y represents CH₂ and Z represents CO, namely the compounds of the following formula (Ia):

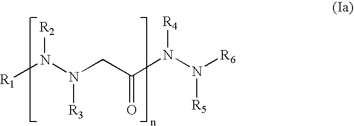

in which n, and R₁ to R₆ are as defined above.

Compounds of formula (Ia) particularly preferred for use within the scope of the present invention are those of the following formulae:

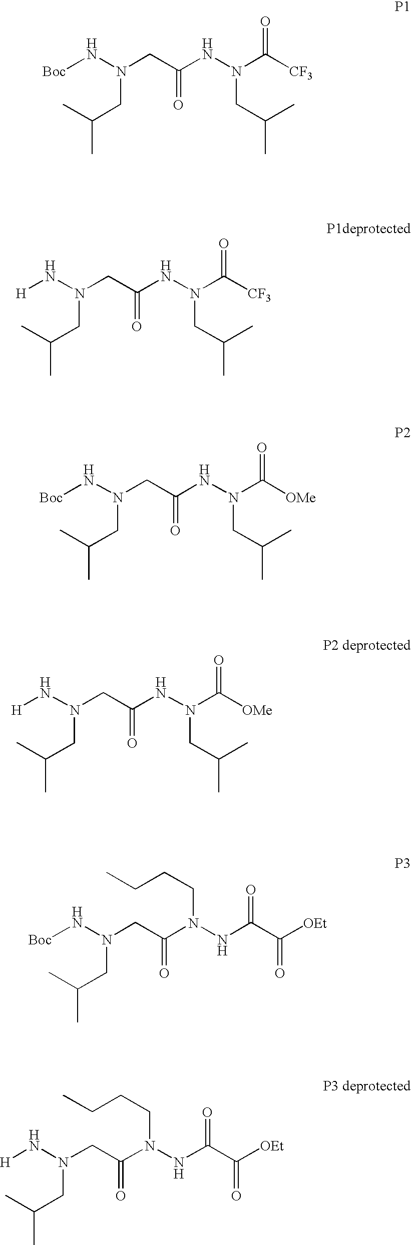

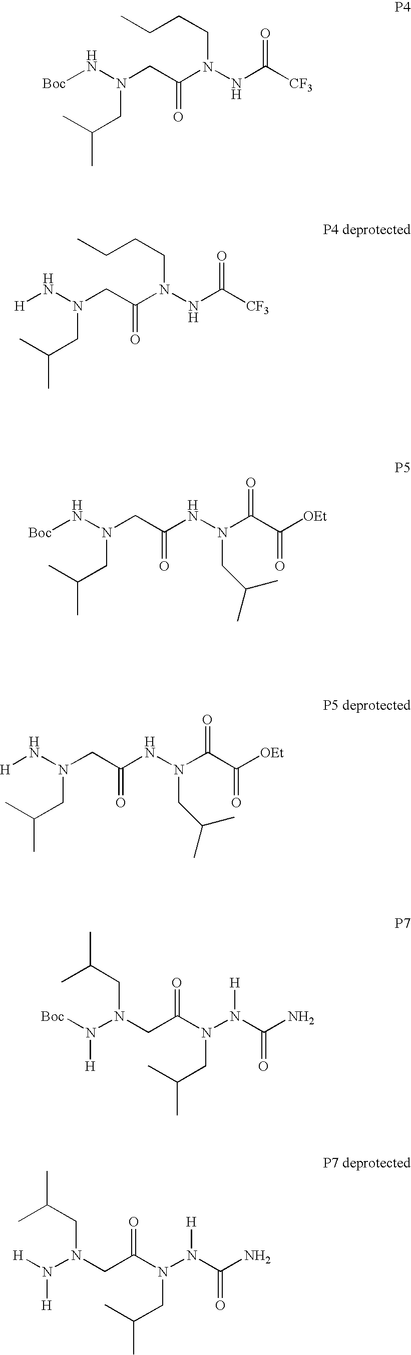

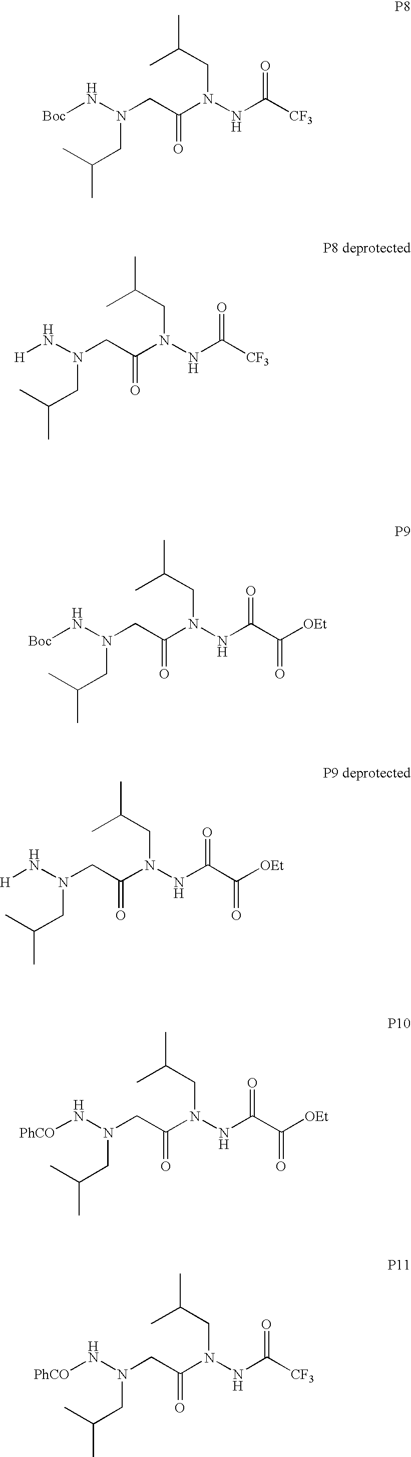

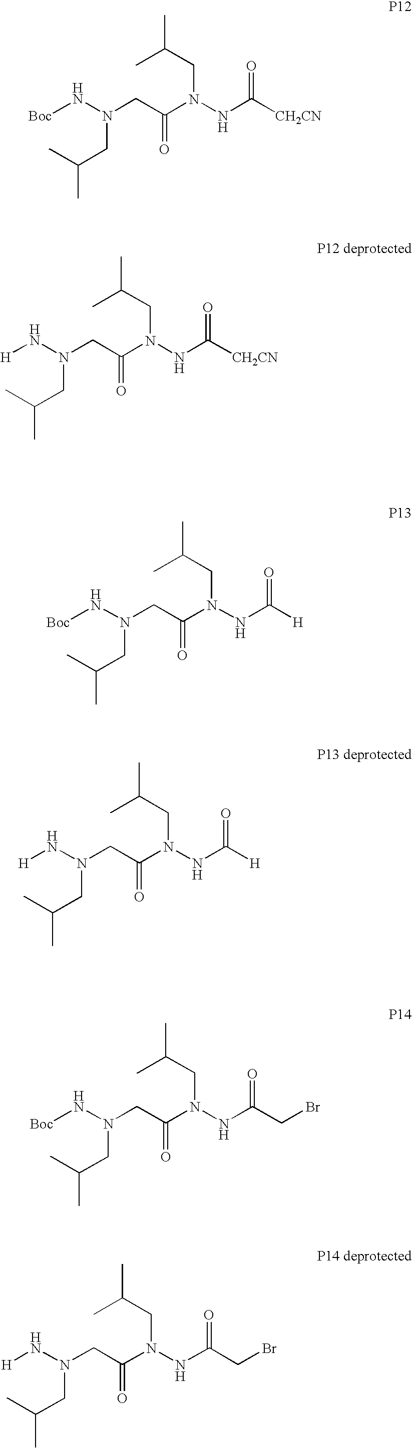

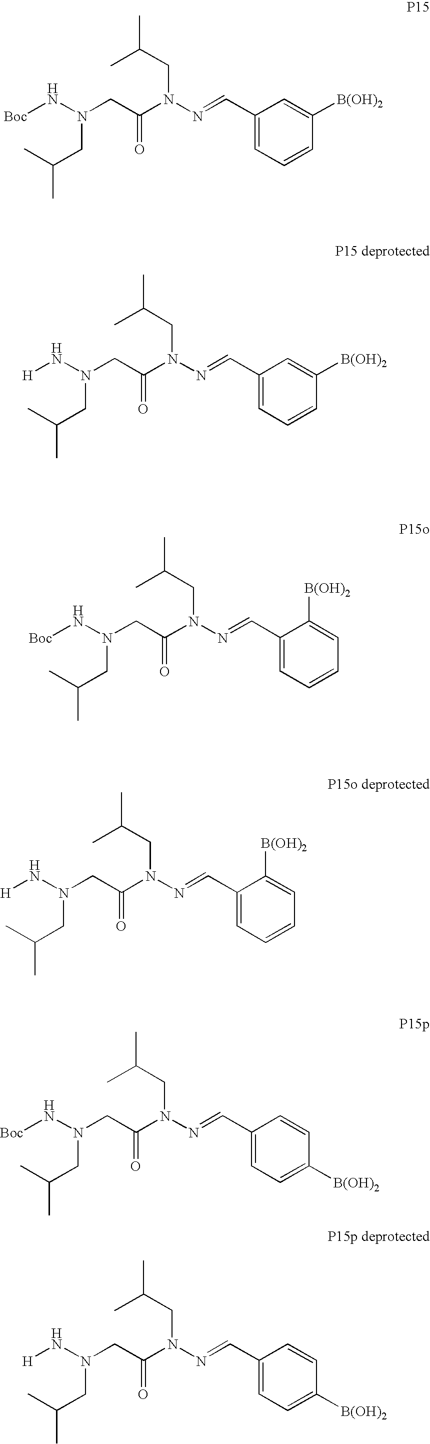

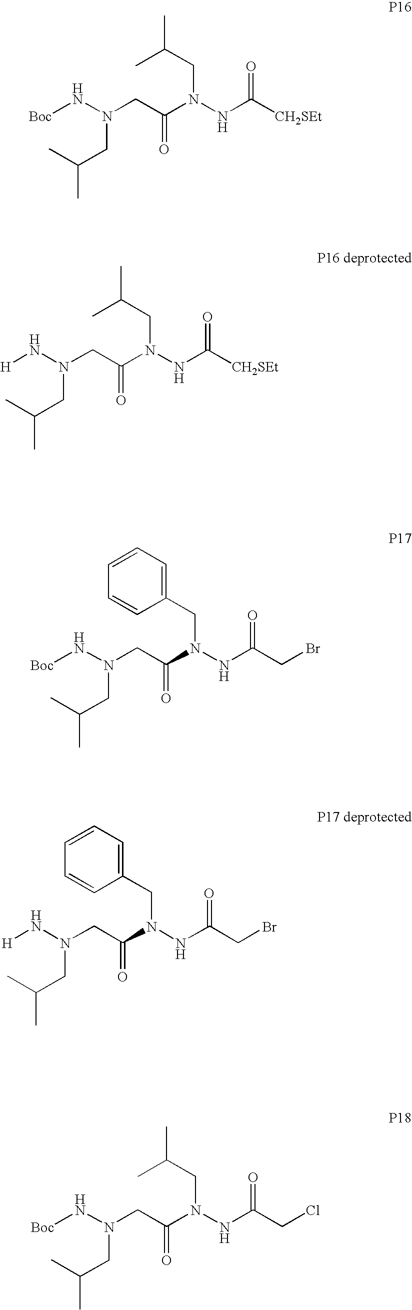

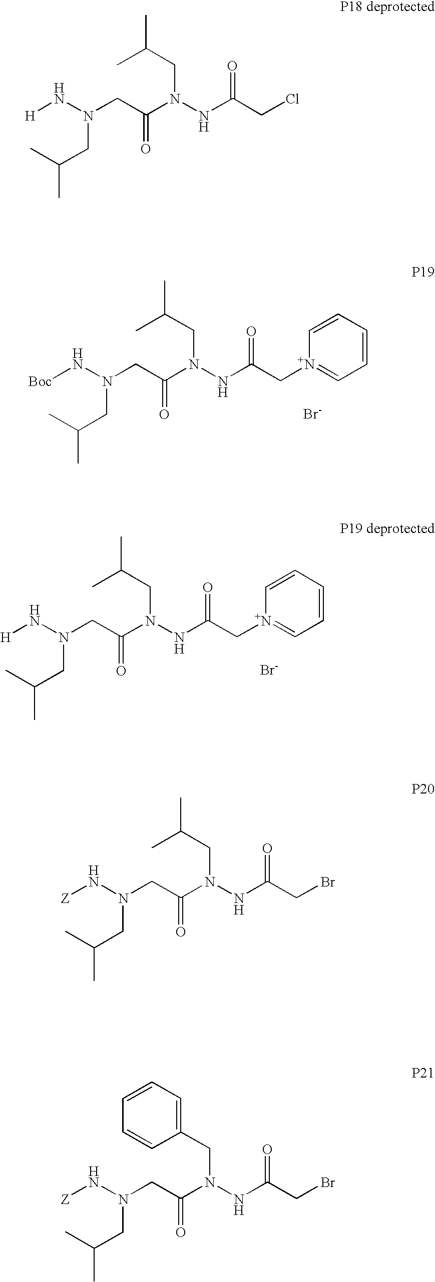

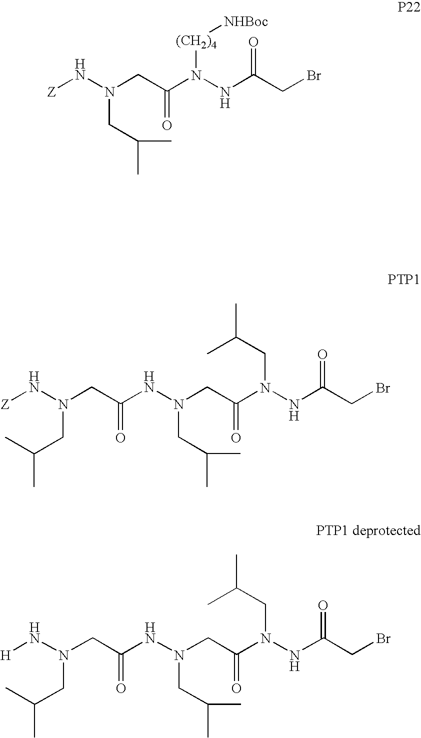

the deprotected compound being in the form of a salt, such as a trifluoroacetate salt.

A more particular subject of the invention is the abovementioned use of the compounds of formula (Ia) corresponding to the following formulae:

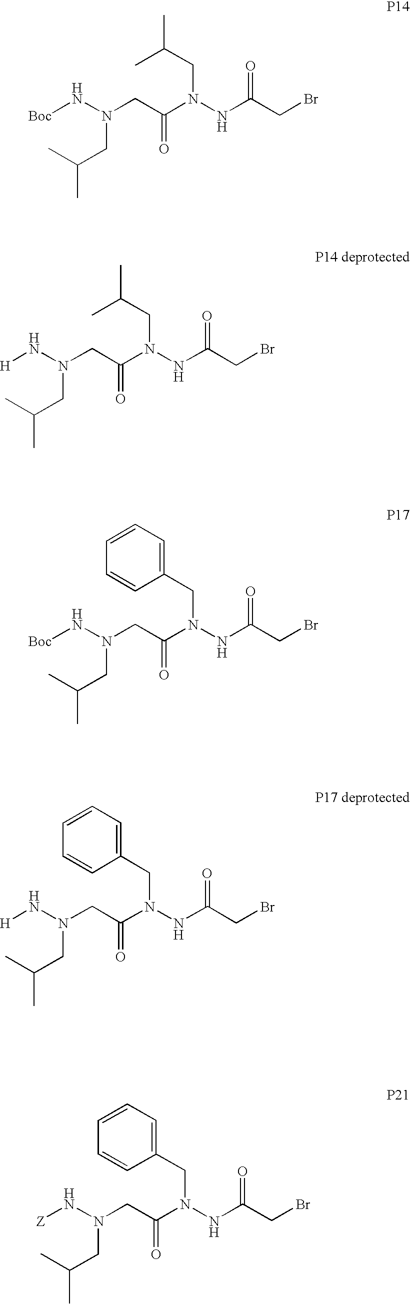

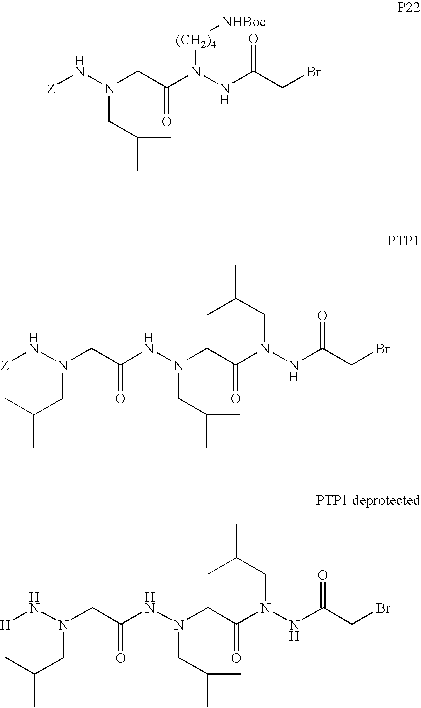

the deprotected compounds being in the form of a salt, such as a trifluoroacetate salt.

A subject of the invention is also the abovementioned use of the compounds of general formula (I), in which Y represents CO and Z represents CH₂, namely the compounds of the following formula (Ib):

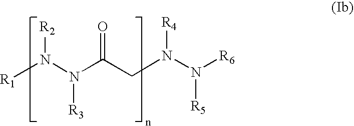

in which n, and R₁ to R₆ are as defined above.

A more particular subject of the invention is the abovementioned use of compounds of formula (Ib) defined above in which:

n represents 1,

R₁ represents a Z group, or H, except that, when R₁ represents H, this is presented in the form of a salt, such as a trifluoroacetate salt of formula CF₃CO₂⁻, H₃N⁺—,

one of R₂ or R₃ represents H, whilst the other represents an alkyl group as defined above, in particular an isobutyl group,

R₄ represents an alkyl group as defined above, in particular an isobutyl group, or a —CH₂C₆H₅, or (CH₂)₄—NH₂, or —CH₂)₄—NHBoc group,

R₅ represents H, and R₆ represents a group of formula —COR as defined above,

or R₅ in combination with R₆ represents a group of formula

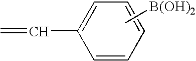

Compounds of formula (Ib) particularly preferred for use within the scope of the present invention are those of the following formulae:

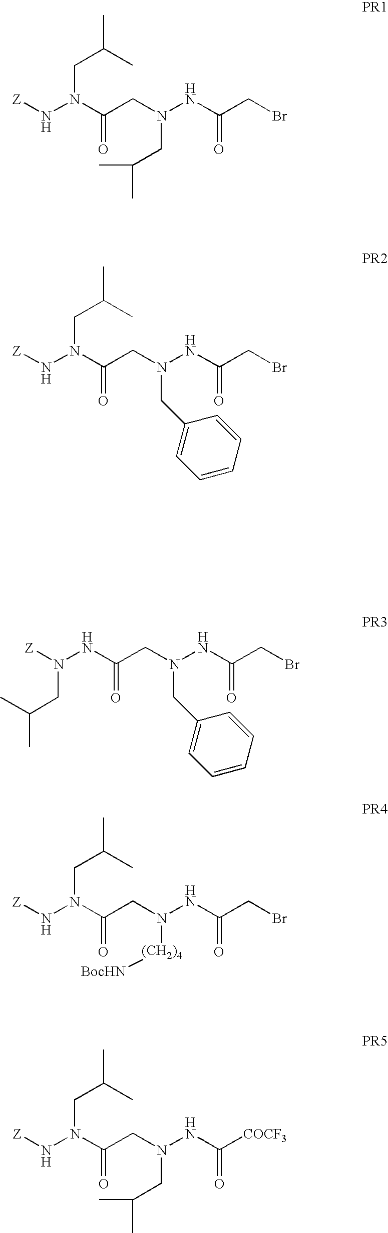

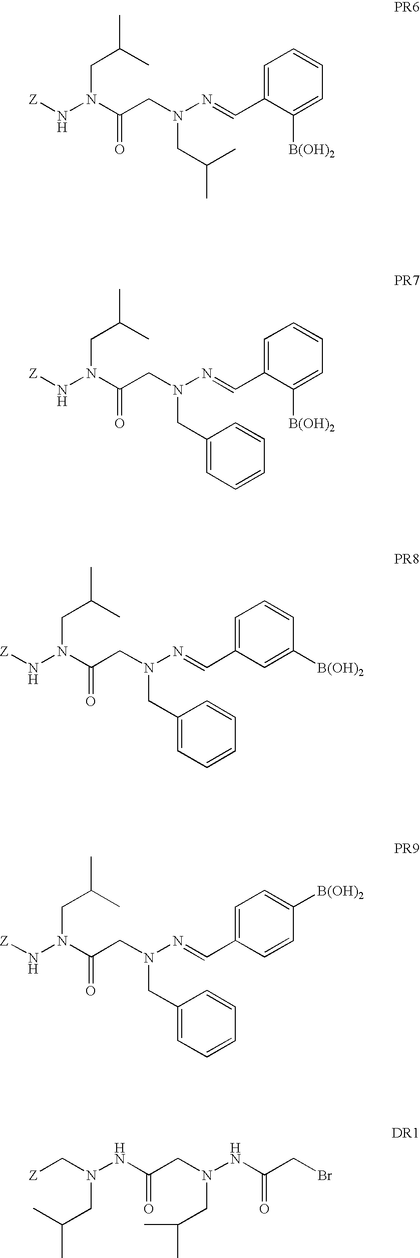

A more particular subject of the invention is the use of the compounds defined above, for the preparation of a medicament intended for the treatment of cancers such as liver, colon, breast cancers, by inducing the entry into apoptosis of the cancerous cells by inhibition of the proteasome functions.

A subject of the invention is also the compounds of the abovementioned general formula (I), and more particularly those of formula (Ia) and (Ib) defined above.

A more particular subject of the invention is the compounds of general formula (Ia) in which:

n=1,

R₅ represents H, and R₆ represents a —COR or —CH₂COR group in which R represents a —CH₂X group, X representing a halogen atom such as Cl or Br, or a pyridinium group,

R₁ to R₄ are as defined above.

A more particular subject of the invention is the abovementioned compounds of formula (Ia), corresponding to the following formulae:

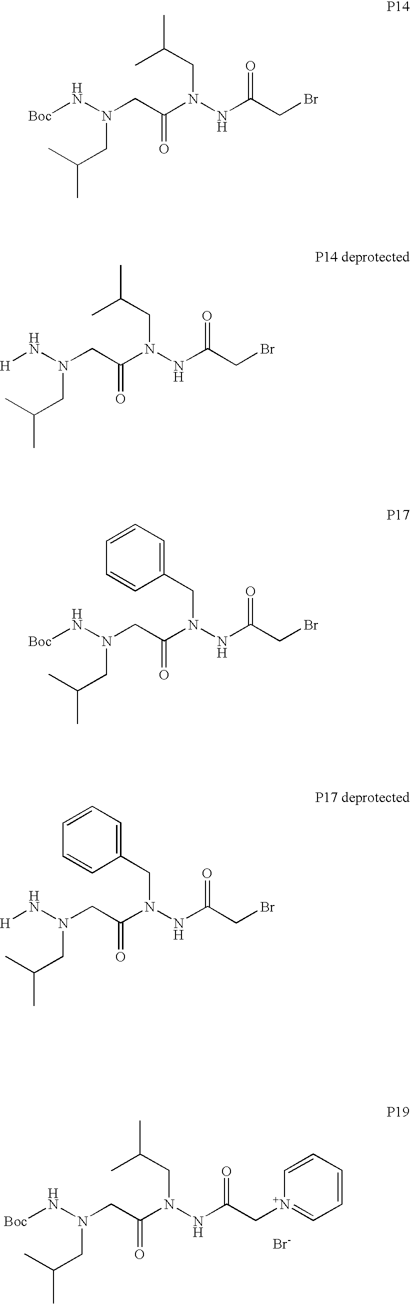

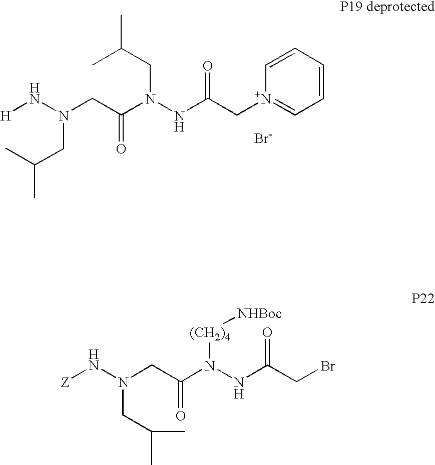

the deprotected compounds being in the form of a salt, such as a trifluoroacetate salt.

The invention also relates to the compounds of general formula (Ia) in which:

n=2,

one of R₄ or R₅ represents H, whilst the other represents an alkyl group as defined above,

R₁, R₂, R₃ and R₆, are as defined above.

A more particular subject of the invention is the abovementioned compounds of formula (Ia) corresponding to the following formulae:

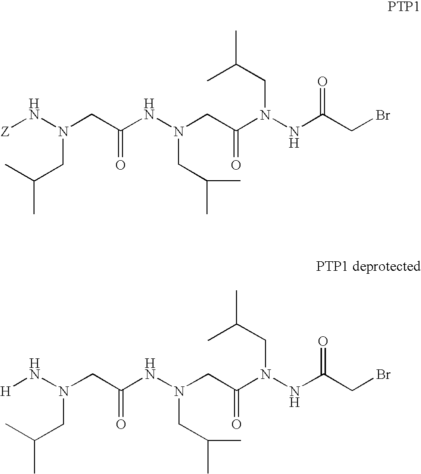

the deprotected compounds being in the form of a salt, such as a trifluoroacetate salt.

A subject of the invention is also the compounds of general formula (Ib) defined above.

The invention thus relates more particularly to the compounds of formula (Ib) defined above in which:

n represents 1,

R₁ represents a Z group, or H, except that, when R₁ represents H, this is presented in the form of a salt, such as a trifluoroacetate salt of formula CF₃CO₂⁻, H₃N⁺—,

one of R₂ or R₃ represents H, whilst the other represents an alkyl group as defined above, in particular an isobutyl group,

R₄ represents an alkyl group as defined above, in particular an isobutyl group, or a —CH₂C₆H₅, or (CH₂)₄—NH₂, or —CH₂)₄—NHBoc group,

R₅ represents H, and R₆ represents a group of formula —COR as defined above,

or R₅ in combination with R₆ represents a group of formula

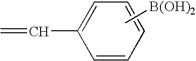

A more particular subject of the invention is the compounds of formula (Ib) corresponding to the following formulae:

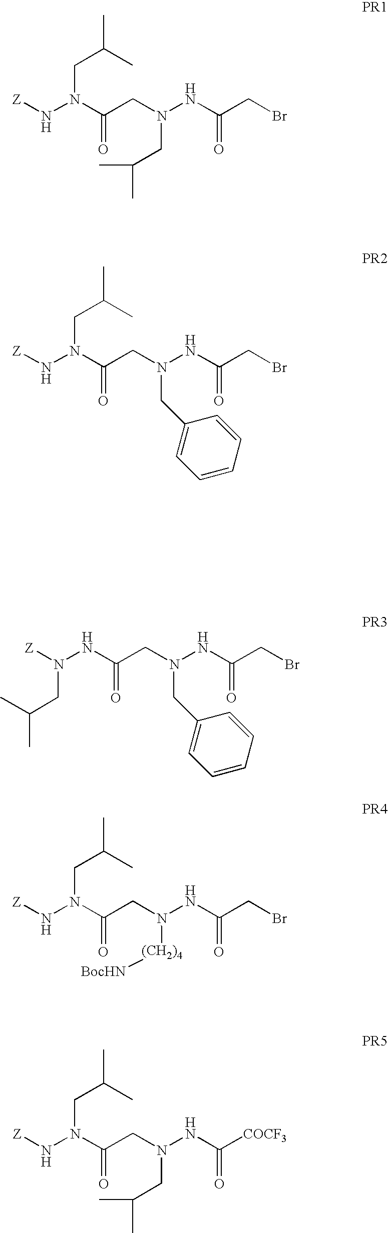

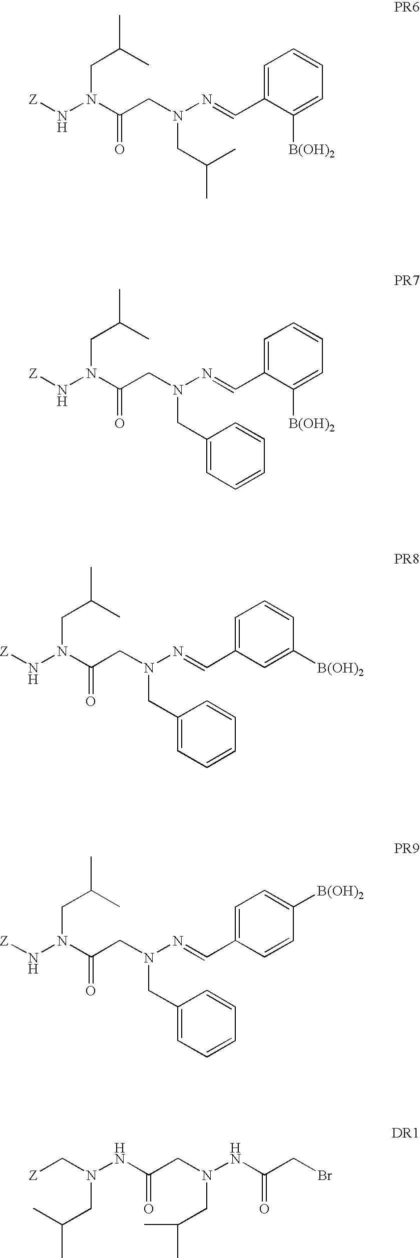

A subject of the invention is also any pharmaceutical composition comprising a compound of formula (I) as defined above in combination with a pharmaceutically acceptable vehicle.

Advantageously, the pharmaceutical compositions of the invention are administered by oral or sub-cutaneous route, and are presented in the form of unit doses of approximately 20 to 50 mg, for a daily administration of approximately 100 mg/kg.

A subject of the invention is also the synthesis process for the compounds of formula (I) defined above, and principally comprising the following stages:

substitution of the compound of formula

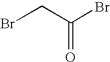

with the products of formula

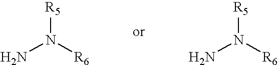

which leads to the obtention of compounds of formulae A and B respectively

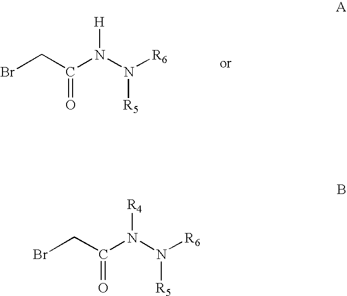

in which R₄, R₅ and R₆ are as defined above,

reaction of the compound of formula

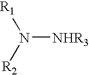

in which R₁ to R₃ are as defined above, with the compounds of the abovementioned formulae A and B, which leads respectively to the compounds of formula (I) hereafter:

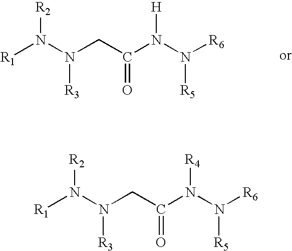

in which R₁ to R₆ are as defined above,

if appropriate, a stage of deprotection by elimination of the R₁ group, in particular according to the deprotection methods described hereafter,

if appropriate, repetition of the abovementioned stages, in order to extend the chain of the compound of formula (I) by the desired number n.

The invention is further illustrated in the following detailed description of the synthesis of compounds of the invention, and of the study of their biological properties.

The ALLN (protease cysteine and proteasome inhibitor) possesses a C-terminal aminoaldehyde as electrophilic group. Other inhibitors of comparable activities have been developed such as the Z-Leu-Norleu-H dipeptide also represented in the diagram. However, it is well known that the amino aldehydes are unstable and are racemized very rapidly, which leads to a loss of activity. The Inventors have therefore synthesized analogues not possessing any centre of asymmetry of fixed configuration in order to obtain an activity of specific inhibition of degradation of the proteins involved in the cycle.

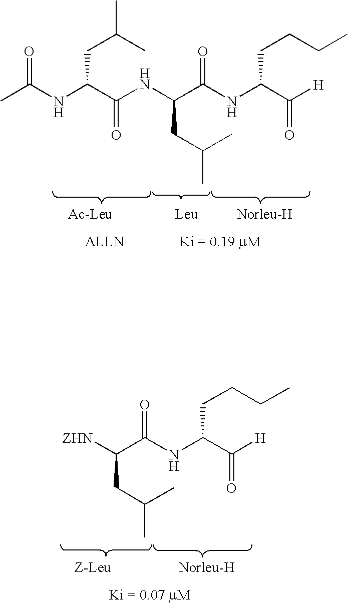

ALLN (N-acetyl-Leucyl-Leucyl-Norleucinal) inhibits the progression of the cell cycle by affecting the G1/S transition and the metaphase-anaphase transition. Strong concentrations of ALLN (>50 μg/ml) produce an extended stop in mitosis whilst lower concentrations result in a slowing-down of the mitosis. The cells can then begin a second cycle.

It is the activity of these peptides involved in cell functions that the Inventors aimed to reproduce via the synthesis of peptidomimetics such as the hydrazinoazapeptoids and the hydrazinopeptoids which are peptide analogues (clearing the maximum physiological barriers, resistance to peptidases).

The peptidomimetics which have been synthesized according to an iterative method are hydrazinoazapeptoids similar to the class of peptoids, azatides and ureapeptoids, in that they possess no centre of asymmetry of fixed configuration. The oligomers of these different families with a peptidomimetic purpose all share the characteristic of having their side chain, mimicking their amino acid homologues, on nitrogen atoms which are isoelectronics of CHαs which gives them considerable conformational freedom. Other potential benefits also result from this, such as a simplification of the synthesis methods (elimination of the stereochemical problems) and a greater resistance of such analogues to the skeletons modified vis-à-vis the action of the peptidases, by modification of the amide bond.

### I) Synthesis of Compounds of Formula (Ia)

The “N-hydrazinoacid” units are introduced in two chemical stages which can be repeated. Moreover, the presence in the hydrazinoazapeptoid units of additional nitrogen atoms compared with the natural peptides offers the possibility, starting with this method, of introducing side chains of various natures on this atom.

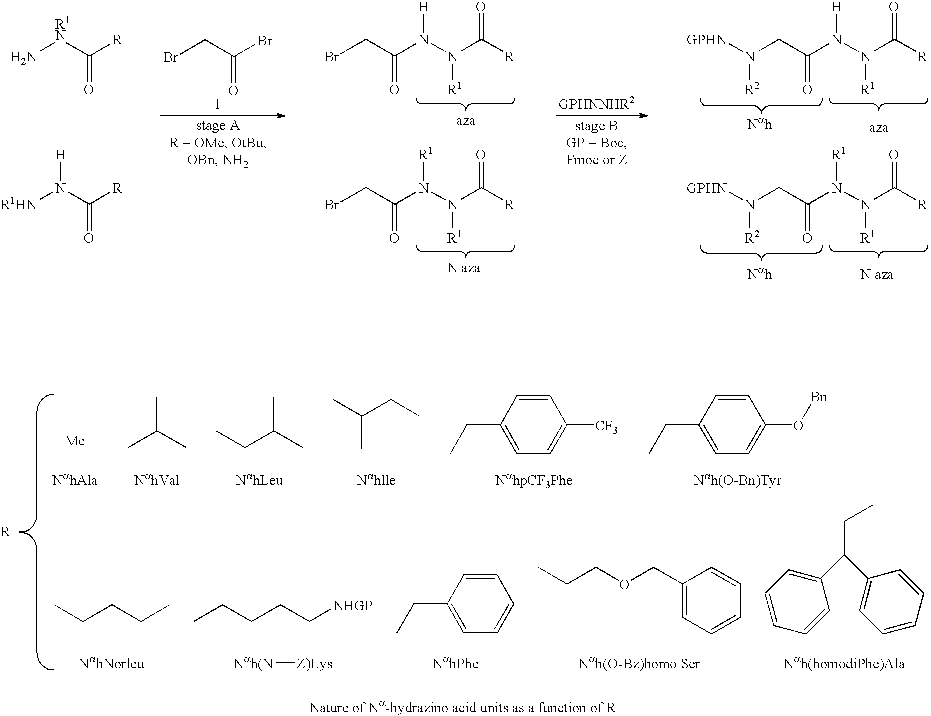

The Inventors have synthesized, according to the above methodology, the compounds combining an aza amino ester unit or, respectively, a C-terminal N-aza amino ester, with a N^{α}-hydrazino acid unit. This makes it possible to obtain a pseudodipeptide skeleton which has the side chains mimicking the amino acids Leucine, Norleucine and Phenylalanine present in the majority of the inhibitors known at present, in various relative positions. Selective cleavage of the protective group at the C-terminal end then makes it possible to refunctionalize and thus to introduce groups capable of interacting with the side chain of the cysteine. The Inventors have thus been able to introduce various functionalities (trifluoroacetyl, ketoester, amide etc.). It is known that such functions are less electrophilic when they are carried by a nitrogen atom but this is moreover a way of increasing the selectivity of an inhibitor vis-à-vis the cysteine proteases (SH more nucleophilic than OH). The different pseudopeptides synthesized are indicated hereafter.

| | | | |
|---|---|---|---|
| P1 | 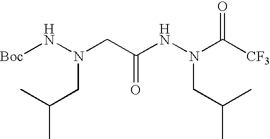 | P2 | 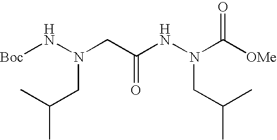 |
| P3 | 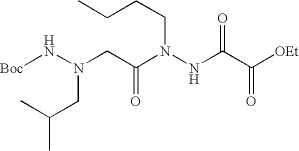 | P4 | 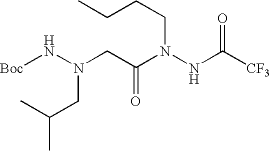 |
| P5 | 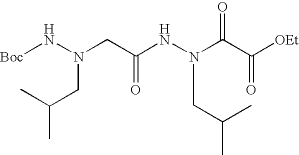 | P6 | 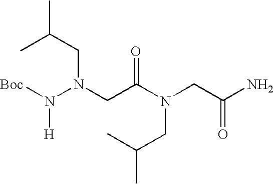 |
| P7 | 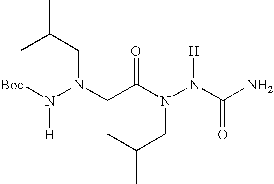 | P8 | 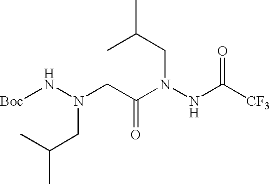 |
| P9 | 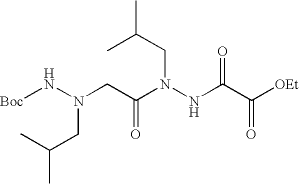 | P10 | 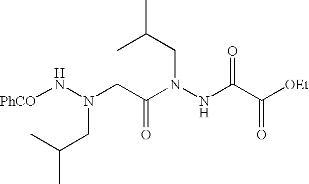 |
| P11 | 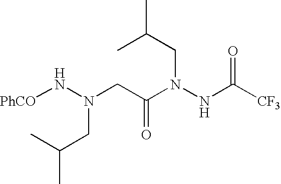 | P12 | 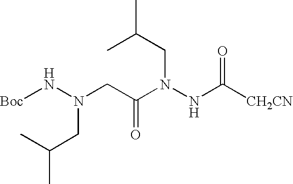 |
| P13 | 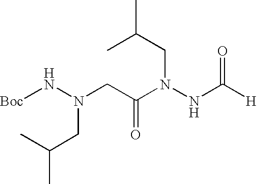 | P14 | 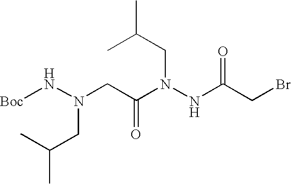 |
| P15 | 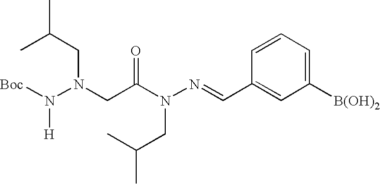 | P16 | 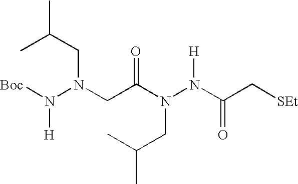 |
| P17 | 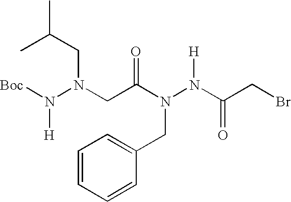 | | |

The Inventors have moreover deprotected the N-terminal end and introduced a new hydrazinopeptoidic unit by repetition of stages A and B in order to obtain a tripeptide analogue (PTP1) closer to the tripeptide structure of the ALLN.

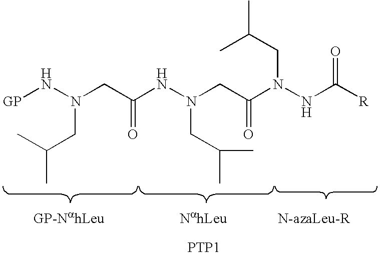

### 1) Bromoacetylated Hydrazines

Bromoacetylation. Bromoacetyl bromide (12 mmol, 1.2 equi) in dichloromethane (10 ml) is added dropwise to a solution cooled down to 0° C, under stirring, of N-protected hydrazine, described in the abovementioned article of Cheguillaume et al., (10 mmol, 1 equi) in dichloromethane (10 ml) and pyridine (12 mmol, 1.2 equi). The mixture is stirred for 5 hours then washed three times using 50 ml of water. The organic phase is dried over sodium sulphate, the solvent is evaporated off under reduced pressure and, according to the nature of the protective group, the product precipitates (Fmoc, Z) or is obtained in the form of an oil (CONH₂).

### Br—CH₂CO-azaLeu-Fmoc

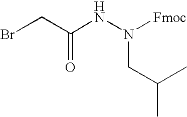

Yield 45%; mp=133° C.; NMR ¹H (CDCl₃) δ (ppm) 0.83 (broad, 6H), 1.73 (broad, 1H), 2.81 (broad, 2H), 3.26 (broad s, 2H), 4.22 (broad, 1H), 4.55 (d, 2H), 7.25-7.77 (m, 8H), 8.28 (s, 1H); NMR ¹³C (CDCl₃) δ (ppm) 19.8 (q), 26.2 (t), 26.7 (d), 47.1 (d), 56.8 (t), 67.5 (t), 119.9 (d), 124.7 (d), 127.1 (d), 127.7 (d), 141.3 (s), 143.5 (s), 155.9 (s), 164.8 (s); Analysis calculated for C₂₁H₂₃N₂O₃Br: C, 58.47; H, 5.34; N, 6.50; Br, 18.56. Found: C, 58.52; H, 5.50; N, 6.64; Br, 17.98.

### Br—CH₂CO-azaNorleu-Fmoc

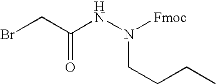

Yield 58%; mp=118° C.; NMR ¹H (CDCl₃) δ (ppm) 1.06 (t, 3H), 1.41 (m, 2H), 1.61 (m, 2H), 3.63 (broad, 2H), 3.81-4.01 (broad s, 2H), 4.38 (t, 1H), 4.69 (d, 2H), 7.44-7.95 (m, 8H), 8.15 (broad s, 1H); NMR ¹³C (CDCl₃) δ (ppm) 14.1 (q), 20.2 (t), 26.7 (t), 29.9 (t), 47.5 (d), 49.9 (t), 68.3 (t), 119.9 (d), 120.4 (d), 125.2 (d), 127.6 (d), 128.2 (d), 141.8 (s), 144.1 (s), 155.9 (s), 165.1 (s); Analysis calculated for C₂₁H₂₃N₂O₃Br: C, 58.47; H, 5.34; N, 6.50; Br, 18.56. Found: C, 58.43; H, 5.16; N, 6.44; Br, 17.90.

### Br—CH₂CO—N-azaLeu-Fmoc

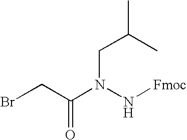

Yield 95%; mp=154° C.; NMR ¹H (CDCl₃) δ (ppm) 0.89 (d, 6H), 1.81 (m, 1H), 3.59 (broad, 2H), 3.93 (broad s, 2H), 4.26 (t, 1H), 4.74 (broad, 2H), 6.84 (s, 1H), 7.32-7.89 (m, 8H); NMR ¹³C (CDCl₃) δ (ppm) 19.9 (q), 26.1 (d), 26.2 (t), 47.4 (d), 54.9 (t), 66.9 (t), 120.2 (d), 125.1 (d), 127.1 (d), 128.2 (d), 141.4 (s), 143.1 (s), 154.5 (s), 169.3 (s); Analysis calculated for C₂₁H₂₃N₂O₃Br: C, 58.47; H, 5.34; N, 6.50; Br, 18.56. Found: C, 56.13; H, 4.93; N, 6.89; Br, 19.44.

### Br—CH₂CO—N-azaNorleu-Fmoc

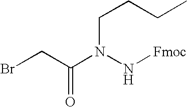

Yield 49%; mp=155° C.; NMR ¹H (CDCl₃) δ (ppm) 0.87 (t, 3H), 1.24 (broad, 2H), 1.36 (broad, 2H), 1.79 (s, 2H), 3.57 (s, 2H), 3.60 (broad s, 2H), 4.20 (t, 1H), 4.67 (broad, 2H), 7.25-7.40 (m, 8H); NMR ¹³C (CDCl₃) δ (ppm) 13.7 (q), 19.7 (t), 26.3 (t), 28.2 (t), 47.3 (d), 47.7 (t), 66.9 (t), 120.1 (d), 124.6 (d), 127.1 (d), 127.9 (d), 141.5 (s), 143.0 (s), 154.7 (s), 168.8 (s); Analysis calculated for C₂₁H₂₃N₂O₃Br: C, 58.47; H, 5.34; N, 6.50; Br, 18.56. Found: C, 58.34; H, 5.34; N, 6.64; Br, 18.20.

### Br—CH₂CO—N-azaLeu-CONH₂

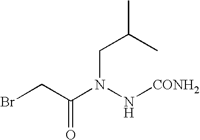

Yield 63%; mp=168° C.; NMR ¹H (DMSO d⁶) δ (ppm) 0.85 (d, 6H), 1.88 (m, 1H), 2.82-3.69 (syst AB, 2H), 3.91-4.21 (syst AB, 2H), 6.21 (s, 2H), 8.58 (s, 1H); NMR ¹³C (CDCl₃) δ (ppm); 20.8 (q), 29.6 (d), 48.3 (t), 55.3 (t), 157.8 (s), 169.6 (s); Analysis calculated for C₇H₁₄N₃O₂Br: C, 33.33; H, 5.56; N, 16.67; Br, 31.75. Found: C, 33.34; H, 5.65; N, 16.92; Br, 31.44.

### Br—CH₂CO—N-azaLeu-CO₂Me

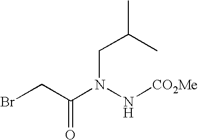

Yield 56%; mp=108° C.; NMR ¹H (DMSO d⁶) δ (ppm) 0.93 (d, 6H), 1.95 (m, 1H), 3.41 (broad, 2H), 3.79 (s, 3H), 3.89 (s, 2H), 8.55 (broad s, 1H); NMR ¹³C (CDCl₃) δ (ppm) 20.3 (q), 26.9 (d), 28.1 (t), 53.2 (q), 55.7 (t), 156.6 (s), 169.5 (s); Analysis calculated for C₈H₁₅N₂O₃Br: C, 35.95; H, 5.62; N, 10.49; Br, 25.96. Found: C, 35.91; H, 5.52; N, 10.50; Br, 25.78.

### Br—CH₂CO—N-azaPhe-Z

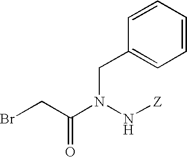

Yield 66%; mp=76° C.; NMR ¹H (CDCl₃) δ (ppm) 3.98 (s, 2H), 4.15-5.40 (syst AB, 2H), 5.19 (s, 2H), 6.97 (s, 1H), 7.34-7.40 (m, 5H); NMR ¹³C (CDCl₃) δ (ppm) 26.7 (t), 51.2 (t), 68.7 (t), 128.8 (d), 129.1 (d), 129.4 (d), 134.8 (d), 135.5 (d), 155.2 (s), 169.4 (s); Analysis calculated for C₁₇H₁₇N₂O₃Br: C, 54.11; H, 3.56; N, 7.43; Br, 21.22. Found: C, 54.56; H, 4.67; N, 7.54; Br, 20.45.

### Br—CH₂CO—N-azaLeu-Z

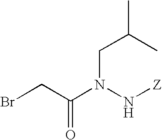

Yield 58%; mp=76° C.; NMR ¹H (CDCl₃) δ (ppm) 0.94 (d, 6H), 1.95 (m, 1H), 2.79-4.18 (broad, 2H), 3.90 (s, 2H), 5.24 (s, 2H), 7.09 (s, 1H), 7.41 (s, 5H); NMR ¹³C (CDCl₃) δ (ppm) 20.3 (q), 26.5 (q), 26.9 (q), 55.5 (t), 68.3 (t), 68.7 (t), 128.5 (d), 128.8 (d), 129.0 (d), 129.2 (d), 135.5 (d), 155.3 (s), 169.9 (s); Analysis calculated for C₁₇H₁₇N₂O₃Br: C, 54.11; H, 3.56; N, 7.43; Br, 21.22.

### Br—CH₂CO—N-azaPhe-Boc

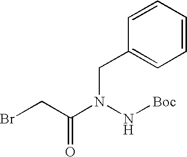

Yield 64%; mp=76° C.; NMR ¹H (CDCl₃) δ (ppm) 1.35 (s, 9H), 3.87 (d, 2H), 4.14-5.20 (broad s, 2H), 6.73 (s, 1H), 7.17-7.27 (m, 5H); NMR ¹³C (CDCl₃) δ (ppm) 27.1 (t), 28.5 (q), 51.3 (t), 83.1 (t), 128.6 (d), 128.7 (d), 129.3 (d), 129.5 (d), 129.7 (d), 135.1 (s), 154.3 (s), 169.5 (s).

### Br—CH₂CO—N-azaLeu-Boc

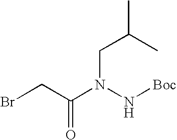

Yield 78%; mp=96° C.; NMR ¹H (CDCl₃) δ (ppm) 0.98 (d, 6H), 1.55 (s, 9H), 1.99 (m, 1H), 2.86-4.22 (broad, 2×2H), 6.80 (s, 1H); NMR ¹³C (CDCl₃) δ (ppm) 20.4 (q), 26.6 (d), 26.9 (t), 28.6 (q), 55.7 (t), 82.9 (s), 154.3 (s), 169.9 (s); Analysis calculated for C₁₇H₁₇N₂O₃Br: C, 54.11; H, 3.56; N, 7.43; Br, 21.22.

### 2) Orthogonally Protected Hydrazinoazapeptoids

Substitution of the bromine atom: α-bromohydrazide (10 mmol, 1 equi) in solution in chloroform (10 ml) is slowly added to a solution under stirring of N-protected hydrazine (25 mmol, 2.5 equi) in chloroform (10 ml). The reaction mixture is taken to reflux under stirring for 24 hours. After cooling down, the medium is washed three times successively using 50 ml of water, 50 ml of 2N HCl, 50 ml of NaHCO₃ and 50 ml of water. The organic phase is dried over sodium sulphate and the solvent is evaporated off under reduced pressure. According to the nature of the two protective groups, the product precipitates slowly from ether in the cold state, or is obtained in the form of a whitish oil.

### Boc-N^{α}hLeu-N-azaLeu-Fmoc

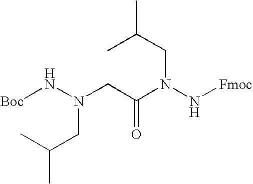

Yield 77% oil; NMR ¹H (CDCl₃) δ (ppm) 0.83 (d, 6H), 0.87 (d, 6H), 1.34 (s, 9H), 1.59 (m, 1H), 1.78 (m, 1H), 2.37 (d, 2H), 3.32 (s, 2H), 3.43 (broad, 2H), 4.14 (t, 1H), 4.43 (d, 2H), 6.01 (s, 1H), 7.15-7.73 (m, 8H), 8.87 (s, 1H).

### Boc-N^{α}hLeu-N-azaLeu-Z

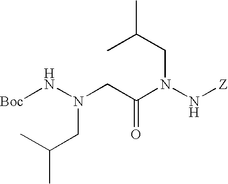

Yield 63%; mp=85° C.; NMR ¹H (CDCl₃) δ (ppm) 0.89 (d, 6H), 0.94 (d, 6H), 1.43 (s, 9H), 1.65 (m, 1H), 1.94 (m, 1H), 2.35 (d, 2H), 3.35-3.50 (broad, 2×2H), 5.20 (s, 2H), 5.60 (s, 1H), 7.39 (s, 5H), 8.96 (s, 1H); NMR ¹³C (CDCl₃) δ (ppm) 20.7 (q), 21.1 (q), 26.5 (d), 26.9 (d), 28.7 (q), 56.1 (t), 62.9 (t), 67.4 (t), 80.8 (s), 128.9 (d), 136.2 (d), 155.9 (s), 156.4 (s), 171.4 (s); Analysis calculated for C₂₃H₃₈N₄O₅: C, 61.31; H, 8.50; N, 12.43, Found: C, 60.80; H, 8.59; N, 12.46.

### Z-N^{α}hLeu-N-azaLeu-Boc

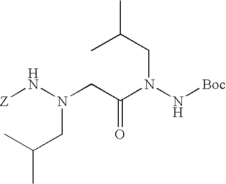

Yield 65%; mp=90° C.; NMR ¹H (CDCl₃) δ (ppm) 0.82 (d, 6H), 0.89 (d, 6H), 1.44 (s, 9H), 1.62 (m, 1H), 1.77 (m, 1H), 2.53 (d, 2H), 3.18-3.51 (syst.AB, 2H), 3.38 (s, 2H), 5.04 (s, 2H), 7.35 (s, 5H), 8.44 (s, 1H), 9.37 (s, 1H); NMR ¹³C (CDCl₃) δ (ppm) 20.3 (q), 20.9 (q), 26.2 (d), 26.4 (d), 28.2 (q), 54.9 (t), 60.1 (t), 65.2 (t), 65.8 (t), 80.6 (s), 128.0 (d), 128.2 (d), 128.7 (d), 137.1 (d), 154.6 (s), 156.3 (s), 171.2 (s); Analysis calculated for C₂₃H₃₈N₄O₅: C, 61.31; H, 8.50; N, 12.43. Found: C, 61.14; H, 8.56; N, 12.48.

### Z-N^{α}hLeu-N-azaPhe-Boc

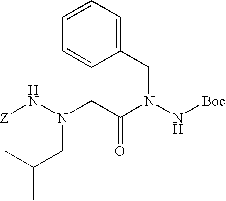

Yield 72%; mp=98° C.; NMR ¹H (CDCl₃) δ (ppm) 1.05 (d, 6H), 1.52 (s, 9H), 1.82 (m, 1H), 2.72 (d, 2H), 3.78 (s, 2H), 4.25-5.55 (broad, 2H), 5.09 (s, 2H), 6.94 (s, 1H), 7.43 (s, 5H), 7.64 (s, 1H); NMR ¹³C (CDCl₃) δ (ppm) 21.1 (q), 26.7 (d), 28.5 (q), 51.4 (t), 60.5 (t), 66.6 (t), 67.2 (t), 82.3 (s), 128.3 (d), 128.5 (d), 128.9 (d), 129.1 (d), 129.7 (d), 135.8 (d), 136.6 (d), 154.4 (s), 156.6 (s), 172.1 (s); Analysis calculated for C₂₆H₃₆N₄O₅: C, 64.46; H, 7.44; N, 11.57. Found: C, 64.20; H, 7.45; N, 11.63.

### 3) Deprotected Pseudopeptoids

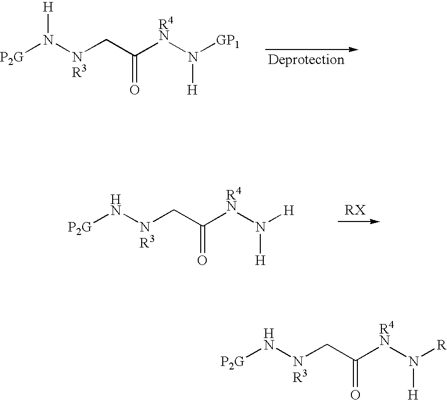

### Selective Deprotection of one of the Ends

For an Fmoc group: Piperidine (20 mmol, 2 equi) in solution in ether (3 ml) is added dropwise to a solution of pseudodipeptoid (10 mmol, 1 equi) in a minimum of ether (5 ml). The reaction mixture is left under stirring for 15 hours. The solvent is evaporated off under reduced pressure and the crude product is recrystallized from ethanol. The white precipitate obtained is an adduct of the reaction, originating from the addition of the piperidine on the fluorene group. After selective recrystallization and filtration of the whole of this adduct, the expected product precipitates slowly from ether in the cold state.

For a Z group: 3 drops of acetic acid and 10% palladium on carbon (Pd/C) (50 mg by mmol of product) are added to a solution under stirring of pseudopeptoid (10 mmol, 1 equi) in ethanol (15 ml). The mixture is placed under a hydrogen atmosphere for 24 hours. The mixture is filtered on celite and dichloromethane is added in order to solubilize the product obtained (white particles in ethanol). The solvents are evaporated off under reduced pressure and the product is obtained in the form of a white solid, insoluble in ether.

For a Boc group: Gaseous HCl, by dehydration of 15 ml of 37% hydrochloric acid on concentrated sulphuric acid (20 ml), is bubbled through a solution under stirring of pseudopeptide (10 mmol, 1 equi) in ether (10 ml). The appearance of the hydrochloride is almost instantaneous and the mixture is left under stirring for 2 hours. The precipitate is then filtered using sintered glass, and washed several times using ether (if the product is to be retained, it is better to leave it in hydrochloride form). The hydrochloride is then solubilized in a 1N NaHCO₃ solution and the free amine is extracted with ether. The organic phase is dried over sodium sulphate, the solvent is evaporated off under reduced pressure. The product is obtained in the form of a thick oil.

### Z-N^{α}hLeu-N-azaLeu-H

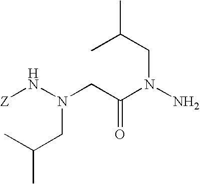

Yield 78%; oil; NMR ¹H (CDCl₃) δ (ppm) 0.93 (d, 2×6H), 1.77 (m, 1H), 2.02 (m, 1H), 2.70 (d, 2H), 3.33 (d, 2H), 3.85-4.13 (broad, 2H), 4.01 (s, 2H), 5.13 (s, 2H), 7.26 (s, 1H), 7.36 (m, 5H); NMR ¹³C (CDCl₃) δ (ppm) 20.6 (q), 21.2 (q), 26.7 (d), 28.6 (d), 56.1 (t), 63.2 (t), 67.2 (t), 81.3 (s), 128.3 (t), 128.6 (t), 128.9 (t), 136.5 (t), 154.7 (t), 157.1 (s), 172.2 (s).

### Boc-N^{α}hLeu-N-azaLeu-H

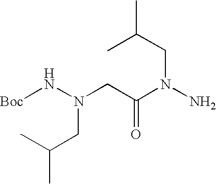

Yield 84%; mp=104° C.; NMR ¹H (CDCl₃) δ (ppm) 0.93 (d, 6H), 0.97 (d, 6H), 1.45 (s, 9H), 1.76 (m, 1H), 2.04 (m, 1H), 2.64 (d, 2H), 3.35 (d, 2H), 3.93 (d, 2H), 4.03 (s, 2H), 6.69 (s, 1H); NMR ¹³C (CDCl₃) δ (ppm) 20.3 (q), 21.1 (q), 26.1 (d), 27.1 (d), 28.7 (q), 57.1 (t), 58.1 (t), 65.8 (t), 79.9 (s), 155.8 (s), 173.2 (s) spectrum having two forms, only the majority form is indicated; Analysis calculated for C₁₅H₃₂N₄O₃: C, 56.96; H, 10.13; N, 17.72. Found: C, 56.73; H, 10.19; N, 17.73.

### Boc-N^{α}hLeu-N-azaNorleu-H

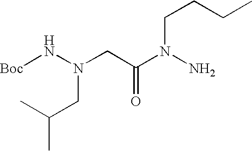

Yield 78%; mp=105° C.; NMR ¹H (CDCl₃) δ (ppm) 0.93 (d, 6H), 0.94 (t, 3H), 1.34 (m, 2H), 1.43 (s, 9H), 1.57 (m, 2H), 1.73 (m, 1H), 2.61 (d, 2H), 3.50 (t, 2H), 3.88 (broad s, 2H), 4.31 (broad s, 2H), 6.79 (broad s, 1H) spectrum having two forms, only the majority form is indicated; NMR ¹³C (CDCl₃) δ (ppm) 14.1 (q), 20.1 (t), 21.0 (q), 26.9 (d), 28.7 (q), 30.2 (t), 49.3 (t), 57.9 (t), 59.6 (t), 65.9 (t), 79.7 (s), 155.8 (s), 172.3 (s) spectrum having two forms, only the majority form is indicated; Analysis calculated for C₁₅H₃₂N₄O₃: C, 56.96; H, 10.13; N, 17.72. Found: C, 56.77; H, 9.99; N, 17.57.

### Boc-N^{α}hLeu-N-azaPhe-H

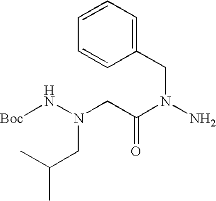

Yield 86%; mp=melting to a paste from 90° C.; NMR ¹H (CDCl₃) δ (ppm) 1.01 (d, 6H), 1.52 (s, 9H), 1.82 (m, 1H), 2.76 (d, 2H), 3.86 (s, 2H), 4.05 (s, 2H), 4.77 (s, 2H), 6.98 (s, 1H), 7.41 (s, 5H); NMR ¹³C (CDCl₃) δ (ppm) 21.1 (q), 27.1 (d), 28.8 (q), 53.2 (t), 59.0 (t), 65.8 (t), 79.8 (s), 127.3 (d), 128.4 (d), 129.0 (d), 129.3 (d), 135.9 (d), 155.7 (s), 173.1 (s); Analysis calculated for C₁₈H₃₀N₄O₃: C, 61.69; H, 8.63; N, 15.99. Found: C, 61.16; H, 8.66; N, 15.71.

### Z-N^{α}hLeu-N-azaPhe-H

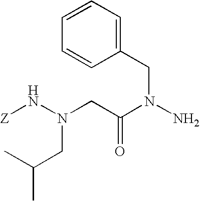

Yield 64%; oil; NMR ¹H (CDCl₃) δ (ppm) 0.96 (d, 6H), 1.77 (m, 1H), 2.50 (s, 1H), 2.75 (d, 2H), 3.73 (s, 2H), 4.02 (s, 2H), 4.66 (s, 2H), 5.11 (s, 2H), 7.30 (m, 2×5 H).

### H-N^{α}hLeu-N-azaLeu-Z

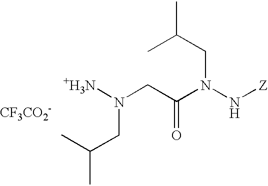

Yield 84%; mp=132° C.; NMR ¹H (CDCl₃) δ (ppm) 0.86 (d, 2×6H), 1.28 (m, 2H), 2.65 (d, 2H), 3.0-4.0 (superimposed signals, 4H), 5.18 (s, 2H), 7.41 (m 5H), 8.81 (broad s, 1H), 9.67 (broad s, 1H); NMR ¹³C (CDCl₃) δ (ppm) 20.2 (q), 20.4 (q), 25.2 (d), 25.9 (d), 54.4 (t), 62.9 (t), 67.1 (t), 128.2 (d), 128.6 (d), 128.9 (d), 136.4 (s), 155.5 (s), 171.3 (s).

### H—N^{α}hPhe-N-azaLeu-Z

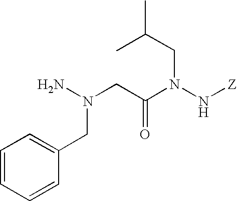

Yield 64%; mp=134° C.; NMR ¹H (CDC;₃) δ (ppm) 0.94 (d, 6H), 1.93 (m, 1H), 3.0-3.55 (superimposed signals, 6H), 3.90 (s, 2H), 5.19 (s, 2H), 7.33 (m, 5H), 7.39 (m, 5H), 7.82 (broad s, 1H); NMR ¹³C (CDCl₃) δ (ppm) 25.3 (q), 31.3 (d), 59.7 (t), 64.2 (t), 69.0 (t), 72.4 (t), 132.4 (d), 133.4 (d), 133.5 (d), 133.6 (d), 133.7 (d), 134.4 (s), 141.0 (s), 143.0 (s), 160.6 (s), 177.7 (s).

### H—N^{α}hPhe-azaLeu-Z

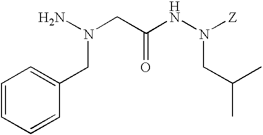

Yield 55%; oil; NMR ¹H (CDCl₃) δ (ppm) 0.95 (d, 6H), 1.93 (m, 1H), 3.2-3.45 (superimposed signals, 6H), 4.01 (s, 2H), 5.21 (s, 2H), 7.31 (m, 10H), 9.01 (broad s, 1H).

### 4) Hydrazinoazapeptoids

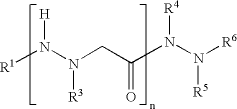

### Functionalization of the C-Terminal End

For the ketone and keto-ester groups. The electrophilic agent (5.5 mmol, 1.1 equi) in solution in ether (5 ml) (trifluoroacetic anhydride and ethyloxalyl chloride) is added dropwise to a solution cooled down to 0° C., under stirring, of pseudodipeptoid (5 mmol, 1 equi) in ether (5 ml) and triethylamine (5.5 mmol, 1.1 equi). The reaction medium is left under stirring for 6 hours. When the triethylammonium salt is insoluble in the ether, it is filtered and the filtrate is evaporated off under reduced pressure; when it is not, the medium is washed three times using 30 ml of water, the organic phase is dried over sodium sulphate and the solvent is evaporated off under reduced pressure. In both cases, the expected product precipitates slowly from ether in the cold state.

### Boc-N^{α}hLeu-N-azaLeu-CF₃: R¹=Boc, R³═R⁴=i-Bu, R⁵═H, R⁶═COCF₃; compound P8

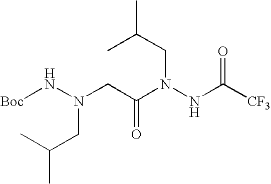

Yield 65%; mp=110° C.; NMR ¹H (CDCl₃) δ (ppm) 0.84 (d, 6H), 0.88 (d, 6H), 1.35 (s, 9H), 1.67 (m, 1H), 1.77 (m, 1H), 2.31 (d, 2H), 3.33 (d, 2H), 3.35 (s, 2H), 5.39 (s, 1H), 11.22 (s, 1H). Analysis calculated for C₁₇H₃₁F₃N₄O₄: C, 49.51; H, 7.52; F, 13.83; N, 13.59. Found: C, 49.30; H, 7.83; F, 13.51; N, 13.86.

### Boc-N^{α}hLeu-N-azaLeu-CO₂Et: R¹=Boc, R³═R⁴=i-Bu, R⁵═H, R⁶═COCO₂Et; compound P9

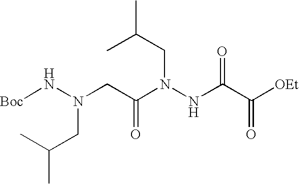

Yield: 54%; NMR ¹H (CDCl₃) δ (ppm) 0.99 (d, 2×6H), 1.49 (t, 3H), 1.51 (s, 9H), 1.78 (m, 1H), 1.94 (m, 1H), 2.49 (d, 2H), 3.48 (s, 2H), 3.54 (d, 2H), 4.45 (q, 2H), 5.91 (s, 1H), 11.06 (s, 1H). Analysis calculated for C₁₉H₃₆N₄O₆: C, 54.81; H, 8.65; N, 13.46. Found: C, 54.85; H, 8.58; N, 13.31.

### PhCO—N^{α}hLeu-N-azaLeu-CF₃: R¹═COPh, R³═R⁴=i-Bu, R⁵═H, R⁶═COCF₃; compound P11

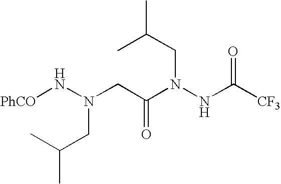

Yield 82%; mp=144° C.; NMR ¹H (CDCl₃) δ (ppm) 0.87 (d, 6H), 0.99 (d, 6H), 1.68 (m, 1H), 1.86 (m, 1H), 2.54 (d, 2H), 3.44 (broad, 2H), 3.51 (s, 2H), 7.46-7.76 (m, 5H), 8.79 (s, 1H), 11.90 (s, 1H); NMR ¹³C (CDCl₃) δ (ppm) 20.8 (q), 21.1 (q), 26.5 (d), 27.3 (d), 55.9 (t), 63.7 (t), 67.5 (t), 127.8 (d), 129.1 (d), 132.1 (d), 132.9 (d), 155.9 (s), 156.7 (s), 168.8 (s), 169.8 (s); Analysis calculated for C₁₉H₂₇F₃N₄O₃: C, 54.81; H, 6.49; F, 13.46; N, 13.70. Found: C, 55.15; H, 6.53; F, 13.28; N, 13.61.

### PhCO—N^{α}hLeu-N-azaLeu-CO₂Et: R¹═COPh, R³═R⁴=i-Bu, R⁵═H, R⁶═COCO₂Et; compound P10

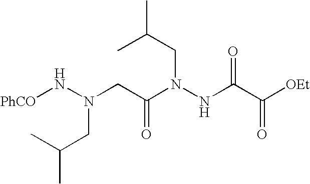

Yield 48%; NMR ¹H (CDCl₃) δ (ppm) 0.77 (d, 6H), 0.86 (d, 6H), 1.34 (t, 3H), 1.70 (m, 1H), 1.76 (m, 1H), 2.47 (d, 2H), 3.35 (d, 2H), 3.49 (s, 2H), 4.31 (q, 2H), 7.33-7.67 (m, 5H), 7.83 (s, 1H), 11.16 (s, 1H); Analysis calculated for C₂₁H₃₂N₄O₅: C, 60.00; H, 7.62; N, 13.33. Found: C, 59.69; H, 7.69; N, 12.96.

### Boc-N^{α}hLeu-N-azaNorleu-CF₃: R¹=Boc, R³=i-Bu, R⁴=n-Bu, R⁵═H, R⁶═COCF₃; compound P4

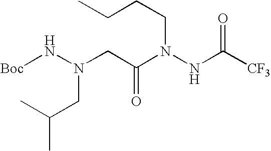

Yield 68%; mp=melting to a paste from 90° C.; NMR ¹H (CDCl₃) δ (ppm) 0.80 (d, 6H), 0.86 (t, 3H), 1.24 (m, 2H), 1.35 (s, 9H), 1.45 (m, 2H), 1.65 (m, 1H), 2.32 (d, 2H), 3.34 (s, 2H), 3.49 (broad, 2H), 5.73 (s, 1H), 11.22 (s, 1H); NMR ¹³C (CDCl₃) δ (ppm) 14.1 (q), 20.3 (t), 20.9(q), 26.3 (d), 28.5 (q), 29.3 (t), 48.2 (t), 64.1 (t), 67.7 (t), 81.6 (s), 113.2 (q), 156.2 (q), 157.3 (s), 168.5 (s); Analysis calculated for C₁₇H₃₁F₃N₄O₄: C, 49.52; H, 7.52; F, 13.84; N, 13.59. Found: C, 49.68; H, 7.68; F, 13.77; N, 13.55.

### Boc-N^{α}hLeu-N-azaNorleu-CO₂Et: R¹═COPh, R³=i-Bu, R⁴=n-Bu, R⁵═H, R⁶═COCO₂Et; compound P3

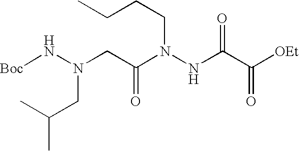

Yield 55%; mp=105° C.; NMR ¹H (CDCl₃) δ (ppm) 0.83 (d, 6H), 0.85 (t, 3H), 1.17-1.41 (m, 2×2H), 1.30 (t, 3H), 1.32 (s, 9H), 1.61 (m, 1H), 2.30 (d, 2H), 3.34 (s, 2H), 3.46 (t, 2H), 4.24 (q, 2H), 5.93 (s, 1H), 11.01 (s, 1H); NMR ¹³C (CDCl₃) δ (ppm) 14.1 (q), 14.2 (q), 20.3 (t), 21.0 (q), 26.4 (d), 28.6 (q), 29.3 (t), 48.1 (t), 63.1 (t), 63.6 (t), 67.4 (t), 80.9 (s), 155.7 (s), 156.6 (s), 159.5 (s), 169.1 (s); Analysis calculated for C₁₉H₃₆N₄O₆: C, 54.81; H, 8.65; N, 13.46. Found: C, 54.62; H, 8.81; N, 13.48.

### Boc-N^{α}hLeu-azaLeu-CF₃: R¹=Boc, R³═R⁵=i-Bu, R⁴═H, R⁶═COCF₃; compound P1

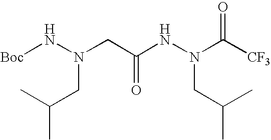

Yield 73%; mp=105° C.; NMR ¹H (CDCl₃) δ (ppm) 0.87 (d, 2×6H), 1.37 (s, 9H), 1.57 (m, 1H), 1.88 (m, 1H), 2.50 (broad, 2H), 3.07-3.86 (syst.AB, 2H), 3.42 (s, 2H), 5.81 (s, 1H), 10.70 (s, 1H); NMR ¹³C (CDCl₃) δ (ppm) 20.1 (q), 20.7 (q), 26.1 (d), 26.9 (d), 28.4 (q), 56.2 (t), 62.3 (t), 69.1 (t), 81.8 (s), 119.3 (q), 157.3 (d, s), 158.5 (q), 170.1 (s); Analysis calculated for C₁₇H₃₁F₃N₄O₄: C, 49.52; H, 7.52; F, 13.84; N, 13.59. Found: C, 49.77; H, 7.80; F, 13.42; N, 13.88.

### Boc-N^{α}hLeu-azaLeu-CO₂Et: R¹=Boc, R³═R⁵=i-Bu, R⁴═H, R⁶═COCO₂Et; compound P5

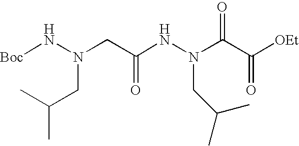

Yield 56%; mp=147° C.; NMR ¹H (CDCl₃) δ (ppm) 0.86 (d, 6H), 0.90 (d, 6H), 1.24 (t, 3H), 1.38 (s, 9H), 1.57 (m, 1H), 1.85 (m, 1H), 2.47 (d, 2H), 3.38 (s, 2H), 3.40 (d, 2H), 4.18 (q, 2H), 5.75 (s, 1H), 10.40 (s, 1H); NMR ¹³C (CDCl₃) δ (ppm) 14.3 (q), 20.2 (q), 20.8 (q), 26.3 (d), 26.8 (d), 28.5 (q), 62.2 (t), 62.7 (t), 68.8 (t), 81.7 (s), 157.1 (s), 162.5 (s), 163.6 (s), 169.8 (s); Analysis calculated for C₁₉H₃₆N₄O₆: C, 54.81; H, 8.65; N, 13.46. Found: C, 54.26; H, 8.49; N, 13.04.

### Boc-N^{α}hLeu-N-azaLeu-CONH₂: R¹=Boc, R³═R⁴=i-Bu, R⁵═H, R⁶═CONH₂; compound P7

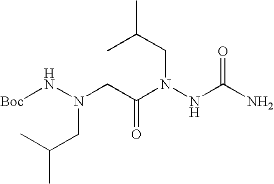

Yield 85%; mp=138° C.; NMR ¹H (CDCl₃) δ (ppm) 0.96 (d, 6H), 0.97 (d, 6H), 1.47 (s, 9H), 1.82 (m, 1H), 2.01 (m, 1H), 2.53 (broad d, 2H), 3.47 (broad s, 2H), 3.58 (broad s, 2H), 5.37 (broad s, 2H), 6.24 (broad s, 1H), 8.69 (broad s, 1H); NMR ¹³C (CDCl₃) δ (ppm); Analysis calculated for C₁₆H₃₃N₅O₄: C, 53.48; H, 9.19; N, 19.50. Found: C, 53.27; H, 9.23; N, 19.39.

For the borylated group: The borylated aldehyde (5.5 mmol, 1.1 equi) in solution in ether (10 mL) is added, by small fractions, to a solution under stirring of pseudopeptoid (5 mmol, 1 equi) in 5 ml of ether. A white precipitate forms instantaneously, but the medium is left under stirring for 1 hour. The white precipitate is filtered using sintered glass and is washed several times with ether.

### Boc-N^{α}hLeu-N-azaLeu-CH-Ph-o-B(OH)₂: R¹=Boc, R³═R⁴=i-Bu, R⁵, R⁶═(HO)₂Bo-(C₆H₄)CH═; compound P15o

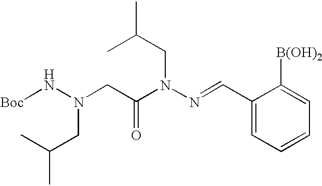

Yield 94%; mp=159° C.; NMR ¹H (DMSO d⁶) δ (ppm) 0.79 (d, 6H), 0.82 (d, 6H), 1.29 (s, 9H), 1.56 (m, 1H), 1.97 (m, 1H), 2.55 (d, 2H), 3.68 (d, 2H), 4.06 (s, 2H), 7.24-7.51(m, 1H+3H), 7.76 (d, 1H), 8.16 (s, 2H), 8.26 (s, 1H); NMR ¹³C (DMSO d⁶) δ (ppm) 20.3 (q), 20.9 (q), 24.9 (d), 26.5 (d), 28.5 (q), 46.9 (t), 58.6 (t), 64.7 (t), 78.5 (s), 126.1 (d), 128.8 (d), 129.3 (d), 134.1 (d), 136.7 (d), 138.1 (s), 142.5 (s), 154.8 (s), 171.7 (s); NMR ¹¹B (DMSO d⁶/Et₂OBF₃) δ (ppm) 30 (broad s); Analysis calculated for C₂₂H₃₇N₄O₅B: C, 58.93; H, 8.32; N, 12.50; B, 2.41. Found: C, 58.64; H, 8.45; N, 12.33; B, 2.16.

### Boc-N^{α}hLeu-N-azaLeu-CH-Ph-p-B(OH)₂: R¹=Boc, R³═R⁴=i-Bu, R⁵, R⁶═(HO)₂Bp-(C₆H₄)CH═; compound P15p

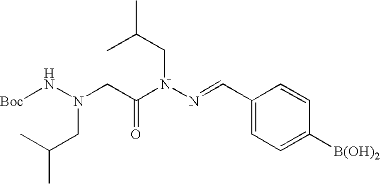

Yield 96%; mp=186° C.; NMR ¹H (DMSO d⁶) δ (ppm) 0.83 (d, 2×6H), 1.31 (s, 9H), 1.59 (m, 1H), 1.95 (m, 1H), 2.58 (d, 2H), 3.76 (d, 2H), 4.07 (s, 2H), 7.46 (s, 1H), 7.64-7.81 (syst AB, 4H), 7.93 (s, 1H), 8.10 (s, 2H); NMR ¹³C (DMSO d⁶) δ (ppm) 20.3 (q), 20.9 (q), 25.1 (d), 26.5 (d), 28.4 (q), 46.5 (t), 58.7 (t), 64.8 (t), 78.5 (s), 126.3 (d), 134.7 (d), 136.1 (d), 136.5 (s), 140.5 (s), 154.8 (s), 171.6 (s); NMR ¹¹B (DMSO d⁶/Et₂OBF₃) δ (ppm) 30 (broad s); Analysis calculated for C₂₂H₃₇N₄O₅B: C, 58.93 H, 8.32; N, 12.50; B, 2.41. Found: C, 58.69; H, 8.32; N, 12.50; B, 2.45.

### Boc-N^{α}hLeu-N-azaLeu-CH-Ph-m-B(OH)₂: R¹=Boc, R³═R⁴=i-Bu, R⁵, R⁶═(HO)₂Bm-(C₆H₄)CH═; compound P15

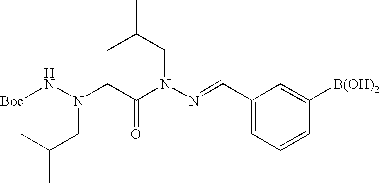

Yield 92%; mp=melting to a paste from 110° C.; NMR ¹H (DMSO d⁶) δ (ppm) 0.92 (d, 6H), 0.95 (d, 6H), 1.41 (s, 9H), 1.54 (m, 1H), 1.68 (m, 1H), 2.68 (d, 2H), 4.00 (broad, 2H), 4.13 (s, 2H), 7.45 (broad, 1H), 7.84 (s, 1H), 7.88 (s, 1H), 8.05 (s, 1H), 8.17 (s, 1H), 8.23 (s, 2H); Analysis calculated for C₂₂H₃₇N₄O₅B: C, 58.93; H, 8.32; N, 12.50; B, 2.41.

For the acetylated groups: Bromoacetyl bromide (6 mmol, 1.2 equi) in dichloromethane (5 mL)is added dropwise to a solution cooled down to 0° C., under stirring, of deprotected hydrazinoazapeptoid (5 mmol, 1 equi) in dichloromethane (10 mL) and pyridine (6 mmol, 1.2 equi). The mixture is stirred for 5 hours then washed three times with 50 ml of water. The organic phase is dried over sodium sulphate, the solvent is evaporated off under reduced pressure. The product precipitates slowly from ether in the cold state.

### Boc-N^{α}hLeu-N-azaLeu-CH₂Br: R¹=Boc, R³═R⁵=i-Bu, R⁴═H, R⁶═COCH₂Br; compound P14

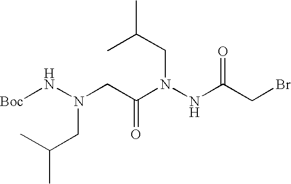

Yield 58%. The product is presented in the form of a foam; NMR ¹H (CDCl₃) δ 0.95 (d, 6H, J=6.5 Hz), 0.98 (d, 6H, J=6.5 Hz), 1.48 (s, 9H), 1.75 (m, 1H), 1.91 (m, 1H), 2.51 (d, 2H, J=7 Hz), 3.46 (d, 2H, J=7 Hz), 3.49 (s, 2H), 3.89 (s, 2H), 5.79 (s, 1H), 10.39 (s, 1H);

### Boc-N^{α}hLeu-N-azaPhe-COCH₂Br: R¹=Boc, R³=i-Bu, R⁴═CH₂Ph, R⁵═H, R⁶═COCH₂Br; compound P17

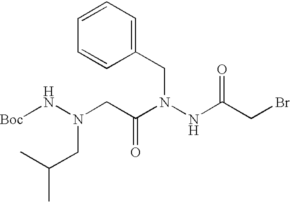

Yield 62%; mp=135° C.; NMR ¹H (CDCl₃) δ 0.81 (d, 6H, J=6.5 Hz), 1.44 (s, 9H), 1.65 (m, 1H), 2.45 (d, 2H, J=6 Hz), 3.52 (s, 2H), 3.85 (s, 2H), 4.85 (broad s, 2H), 5.69 (s, 1H), 7.33 (s, 5H), 10.15 (s, 1H); Analysis calculated for C₂₀H₃₁N₄O₄Br: C, 50.96; H, 6.63; N, 11.89; Br, 16.95. Found: C, 50.87; H, 6.65; N, 11.79; Br, 16.46.

### Z-N^{α}hLeu-N-azaPhe-CH₂Br R¹=Z, R³=i-Bu, R⁴═CH₂Ph, R⁵═H, R⁶═COCH₂Br; compound P21

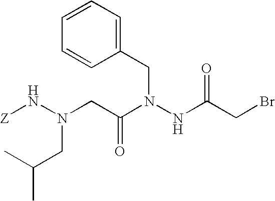

Yield 60%; oil; NMR ¹H (CDCl₃) δ 0.83 (d, 6H, J=6.75 Hz), 1.68 (m, 1H), 2.50 (d, 2H, J=7 Hz), 3.54 (s, 2H), 3.75 (s, 2H), 4.77 (s, 2H), 5.01 (s, 2H), 6.67 (s, 1H), 7.35 (s, 5H), 10.04 (s, 1H).

### Boc-N^{α}hLeu-N-azaLeu-CH₂Cl: R¹=Boc, R³═R⁴=i-Bu, R⁵═H, R⁶═COCH₂Cl; compound P18

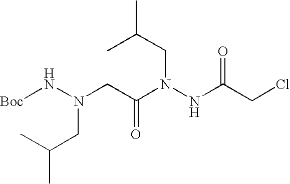

Yield 57%; oil; NMR ¹H (CDCl₃) δ 0.95 (d, 6H, J=6.5 Hz), 0.98 (d, 6H, J=6.5 Hz), 1.47 (s, 9H), 1.75 (m, 1H), 1.88 (m, 1H), 2.48 (d, 2H, J=7 Hz), 3.48 (broad s, 2×2H), 4.10 (s, 2H), 5.63 (s, 1H), 10.34 (s, 1H).

For the pyridinium group: Pyridine (6 mmol, 1,2 equi) is added to a solution under stirring of bromoacetylated pseudopeptoid (5 mmol, 1 equi) in ether (5 ml). The reaction medium is left under stirring for 14 hours at ambient temperature. After evaporation of the solvent under reduced pressure, a foam is obtained containing the product and the excess pyridine. This excess is removed by adding petroleum ether to the foam (pyridine is soluble, but not the pyridinium salt). The petroleum ether is drawn off using a pipette and the operation is repeated three times. The remainder of the petroleum ether is evaporated off under reduced pressure and the product obtained is a foam which is fairly solid when it is dried.

### Boc-N^{α}hLeu-N-azaLeu-Ac-Pyr⁺Br⁻; compound P19

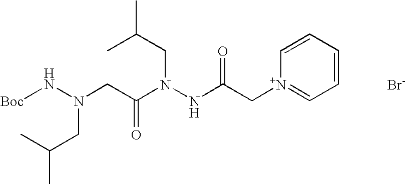

Yield 60%. The product is presented in the form of a foam; NMR ¹H (CDCl₃) δ (ppm) 0.90 (d, 6H), 0.93 (d, 6H), 1.43 (s, 9H), 1.70 (m, 1H), 1.98 (m, 1H), 2.70 (d, 2H), 3.40 (d, 2H), 3.94 (s, 2H), 6.31 (s, 2H), 6.97 (s, 1H), 8.12 (t, 2H), 8.54 (t, 1H), 9.39 (d, 2H), 11.44 (broad s, 1H); NMR ¹³C (CDCl₃) δ (ppm) 19.2 (q), 19.8 (q), 25.3 (d), 25.5 (d), 27.4 (q), 53.6 (t), 57.0 (t), 60.1 (t), 64.1 (t), 78.7 (s), 126.9 (d), 145.0 (d), 145.6 (d), 154.9 (s), 162.9 (s), 171.1 (s).

For the aldehyde group: Formic acid (6 mmol, 2.4 equi) and DCC (5 mmol, 2 equi) are added to a solution cooled down to 0° C., under stirring, of pentafluorophenol (5 mmol, 2 equi) in ether (5 ml). After stirring for ten minutes, the pseudopeptoid (2.5 mmol, 1 equi) is added in solution in 5 ml of chloroform, the reaction medium is left under stirring for 4 hours at ambient temperature. The mixture is then diluted with 20 ml of chloroform and DEEA is added (5 mmol, 2 equi). The medium is washed with 10 ml of 1N HCl, 10 ml of 5% NaHCO₃ and 10 ml of water. The organic phase is dried over sodium sulphate and the solvents are evaporated off under reduced pressure. The product precipitates after having been cooled down with liquid air (paste which solidifies and which is insoluble in ether).

### Boc-N^{α}hLeu-N-azaLeu-H: R¹=Boc, R³═R⁴=i-Bu, R⁵═H, R⁶═CHO; compound P13

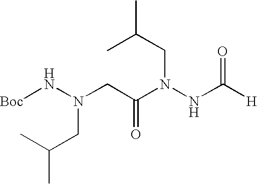

Yield 85%; mp=124° C.; NMR ¹H (CDCl₃) δ (ppm) 0.94 (d, 6H), 0.98 (d, 6H), 1.46 (s, 9H), 1.75 (m, 1H), 1.94 (m, 1H), 2.47 (d, 2H), 3.48 (s, 2H), 3.50 (d, 2H), 5.69 (s, 1H), 8.12 (s, 1H), 10.34 (s, 1H); NMR ¹³C (CDCl₃) δ (ppm) 20.7 (q), 21.0 (q), 26.5 (d), 27.1 (d), 28.7 (q), 55.3 (t), 63.8 (t), 67.0 (t), 81.4 (s), 156.8 (d), 159.8 (s), 169.7 (s): Analysis calculated for C₁₆H₃₂N₄O₄: C, 55.76; H, 9.36; N, 16.27. Found: C, 55.74; H, 9.47; N, 16.18.

### Extension of the Chain by Repetition of Stages A and B; compound PTP1 Z-N^{α}hLeu-N^{α}hLeu-N-azaLeu-CH₂Br

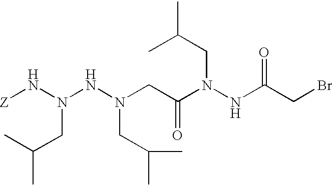

Foam; NMR ¹H (CDCl₃) δ 0.94 (d, 3×6H), 1.68 (m, 2×1H), 1.91 (m, 1H), 2.57 (d, 2×2H), 3.34 (s, 2H), 3.47 (s, 2×2H), 3.85 (s, 2H), 5.13 (s, 2H), 6.16 (s, 1H), 7.36 (m, 5H), 9.31 (s, 1H), 10.84 (s, 1H).

### Z-N^{α}hLeu-N-azaLys-COCH₂Br: R¹=Z, R³=i-Bu, R⁴═(CH₂)₄NHBoc, R⁵═H, R⁶═COCH₂Br. Compound P22

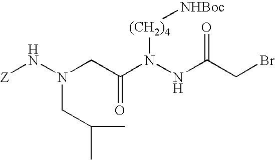

NMR ¹H (CDCl₃) δ 0.83 (d, 6H, J=6.5 Hz), 1.34 (s, 9H+4H), 1.65 (m, 1H), 2.45 (d, 2H, 7.5 Hz), 2.65 (s, 2H), 2.98 (d, 2H, 7.5 Hz), 3.44 (s, 2H), 3.71 (s, 2H), 4.82 (bs, 1H), 5.00 (s, 2H), 7.05 (s, 1H)), 7.23 (m, 5H), 10.2 (bs, 1H). NM ¹³C (CDCl₃) δ (ppm): 20.95 (q), 24.3 (t), 26.4 (t), 27.5 (d), 28.8 (q), 41.2 (t), 47.5 (t), 62.0 (t), 66.1 (t), 67.5 (t), 79.8 (s), 128.2 (d), 128.6 (d), 128.9 (d), 136.3 (s), 157.4 (s), 166.1 (s), 172.3 (s).

### II) Biological Analyses of the Compounds of Formula (Ia)

The molecules synthesized were tested in vitro on the proteolytic activities described of the proteasome, then in vivo on Xenopus (XL2) cell cultures. Knowledge of the cell cycle of XL2 cells has made it possible to carry out synchronization experiments and also to evaluate the duration of each of the phases of the cycle.

In vitro analyses of the inhibitory potentialities of the Hydrazinoazapeptoids synthesized vis-à-vis the enzyme activities of the proteasome.

The inhibitory potentialities of the hydrazinoazapeptoids have been quantified with respect to the chymotrypsin enzyme activities of the purified proteasome. The results are expressed as percentages of activity inhibition.

Measurement of the inhibitory properties of the synthesized compounds on the chymotrypsin catalytic activity of the purified proteasome.

| % | P1 | P2 | P3 | P4 | P5 | P6 | P7 | P8 | P9 | P10 | P11 | P12 | P13 | P14 | P15 | P16 | P17 | ALLN |
|---|---|---|---|---|---|---|---|---|---|---|---|---|---|---|---|---|---|---|
| 2 mM | 29 | 47 | 31 | 29 | 32 | 38 | 23 | 28 | 30 | 42 | 40 | 48 | 35 | 71 | 38 | 34 | 80 | |
| 1 mM | | | | | | | | | | | | | | | | | | 91 |

It will be noted that the compounds P14 and P17 have a particularly useful inhibitory activity. It is possible to inhibit the proteasome activity by 70% with 2 mM of these compounds. 1 mM of ALLN is necessary to inhibit 90% of this activity.

### FACS Analysis

The biological results of the products tested on the XL2 cell cultures are shown in the table below. Dose (from 2 μM to 174 μM) and kinetic (from 0.5 h to 7 h) effects were carried out.

% of cells blocked in G₂/M

| | μM | h | % |
|---|---|---|---|
| Control | | | 4.2 |
| ALLN | 100 | 7 | 53 |
| MG132 | 50 | 7 | 60.6 |
| P1 | 145 | 7 | 9.2 |
| P2 | 160 | 7 | 6.7 |
| P3 | 144 | 4 | 14.9 |
| P3 | 144 | 6 | 15.2 |
| P3 | 144 | 8 | 13.8 |
| P4 | 145 | 4 | 16.5 |
| P4 | 145 | 8 | 13.4 |
| P5 | 144 | 7 | 5.2 |
| P6 | 167 | 7 | 9.2 |
| P7 | 167 | 7 | 12.3 |
| P8 | 146 | 7 | 19.1 |
| P9 | 144 | 7 | 14.1 |
| P10 | 143 | 7 | 23.1 |
| P11 | 144 | 7 | 17.2 |
| P12 | 156 | 7 | 23.5 |
| P13 | 174 | 7 | 20.8 |
| P14 | 137 | 0.5 | 37.9 |
| P14 | 137 | 1 | 50.2 |
| P14 | 2 | 0.5 | 48.2 |
| P14 | 4 | 0.5 | 44.8 |
| P14 | 11 | 0.5 | 47.4 |
| P14 | 22 | 0.5 | 46.2 |
| P14 | 45 | 0.5 | 48.3 |
| P14 | 91 | 0.5 | 50.2 |
| P14 | 137 | 0.5 | 42.8 |
| P15 | 133 | 7 | 35.8 |
| P16 | 143 | 7 | 24.2 |
| P17 | 127 | 0.5 | 42.8 |
| P17 | 127 | 1 | 41.7 |
| P17 | 127 | 2 | 38.3 |
| P17 | 21 | 0.5 | 46.8 |
| P17 | 42 | 0.5 | 47.3 |
| P17 | 85 | 0.5 | 50.9 |

Two inhibitors P14 and P17 have an activity comparable to that of the ALLN, if the percentage of cells blocked in mitosis is analyzed.

The percentage of cells blocked in mitosis is greater than 20% for a good number of these products. The Inventors have therefore improved the bioactivity of the products by the modification of the C-terminal end and by the position of the side chains on the pseudopeptidic skeleton. Moreover, it can be noted that the concentration of P14 necessary, in order to obtain an equivalent blockage in mitosis in the medium is 2 μM.

It is particularly interesting to note that the two inhibitors P14 and P17 are capable of blocking the progression of the cycle and more particularly in mitosis. It can be noted that the concentration of P14 in the medium necessary in order to obtain a blockage in mitosis is 2 μM whereas the concentration of ALLN which allows the blockage of the cells in mitosis is 100 μM.

[image]

### Fluorescence Microscopy Analysis

Observation of the nuclear content makes it possible to determine the different stages of the mitosis. The following results were obtained using 50 cells blocked in mitosis. The first 7 inhibitors were studied using fluorescence microscopy.

| Product | % Prophase | % Metaphase | % Anaphase + Telophase |
|---|---|---|---|
| Control | 45 | 45 | 10 |
| ALLN | 15 | 83 | 3 |
| P1 | 14 | 35 | 51 |
| P2 | 18 | 36 | 46 |
| P3 | 21 | 26 | 53 |
| P4 | 16 | 42 | 42 |
| P5 | 18 | 45 | 37 |
| P6 | 18 | 24 | 58 |
| P7 | 27 | 35 | 38 |

According to the nature of the modifications made to the peptide skeleton, the blockage of the cells occurs at different phases of the cell cycle. It can be noted that the products P6 and P7 do not have the same inhibitory selectivity during the different stages of mitosis. It can thus be seen that P6 mostly blocks cells at the end of mitosis, whereas P7 is shown to be much less selective.

### III) Synthesis of the Compounds of Formula (Ib)

### 1) Retro Hydrazinopeptoids (Inversion of the Hydrazide Bond)

### Functionalization of the N-Terminal End

For the bromo-acetylated groups: Bromoacetyl bromide (6 mmol, 1.2 equi) in dichloromethane (5 mL) is added dropwise to a solution cooled down to 0° C., under stirring of deprotected hydrazinoazapeptoid (5 mmol, 1 equi) in dichloromethane (10 mL) and pyridine (6 mmol, 1.2 equi). The mixture is stirred for 5 hours then washed three times using 50 ml of water. The organic phase is dried over sodium sulphate, the solvent is evaporated off under reduced pressure. The product precipitates slowly from ether in the cold state.

### BrH₂COC—N^{α}hLeu-N-azaLeu-Z: R¹=Z, R²═R⁵═H, R³═R⁴=i-Bu, R⁶═COCH₂Br, compound PR1.

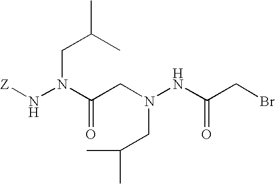

NMR ¹H (CDCl₃) δ 0.80 (2×d, 12H, J=6.6 Hz), 1.56 (m, 1H),1.81 (m, 1H), 2.46 (broad d, 2H), 3.29-3.61 (6H), 5.09 (s, 2H), 7.27 (m, 5H), 8.14 (s, 1H), 8.42 (s, 1H). M⁺ theoretical m/z: 471.16069; found m/z: 471.1613.

### BrH₂COC—N^{α}hPhe-N-azaLeu-Z: R¹=Z, R²═R⁵═H, R³=i-Bu, R⁴═CH₂Ph, R⁶═COCH₂Br. compound PR2.

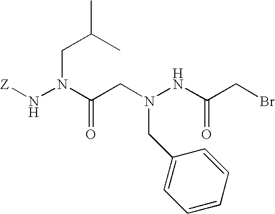

mp=109° C.; NMR ¹H (CDCl₃) δ 0.99 (d, 6H, J=6.6 Hz), 1.72 (m, 1H), 3.1-4.3 (overlapping signals, 8H), 5.24 (s, 2H), 7.44 (m, 10H)), 7.78-8.51 (broad, 2H). NMR ¹³C (CDCl₃) δ (ppm) 20.5 (q), 27.0 (q), 55.9 (t), 57.8 (t), 61.7 (t), 68.3 (t), 68.5 (t), 128.4, 128.8, 129.1, 129.9, 130.3, 135.8, 155.5, 165.4, 172.2 (s). Analysis calculated for C₂₃H₂₉N₄O₄Br: C, 54.66; H, 5.78; N, 11.09; Br, 15.91. Found: C, 54.61; H, 5.80; N, 11.06; Br, 15.98. M^{+:} theoretical m/z: 505.14504; found m/z: 505.1454.

### BrH₂COC—N^{α}hPhe-azaLeu-Z: R¹=Z, R²=i-Bu, R³═R⁵═H, R⁴═CH₂Ph, R⁶═COCH₂Br. compound PR3.

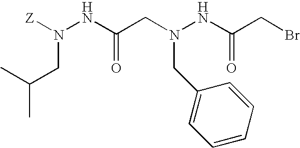

mp=130° C.; NMR 1H (CDCl₃) δ 0.98 (d, 6H, J=6.6 Hz), 1.91 (m, 1H), 3.30, 3.58, 3.80 (overlapping signals, 6H), 4.03 (s, 2H), 5.15 (s, 2H), 7.37 (m, 1H)), 7.46 (s, 1H), 9.69 (s, 1H). Analysis calculated for C₂₃H₂₉N₄O₄Br: C, 54.66; H, 5.78; N, 11.09; Br, 15.91. Found: C, 54.55; H, 5.77; N, 11.19; Br, 15.92.

### BrH₂COC—N^{α}hLys(Boc)-azaLeu-Z: R¹=Z, R²═R⁵═H, R³=i-Bu, R⁴═(CH₂)₄Lys(Boc), R⁶═COCH₂Br. Compound PR4.

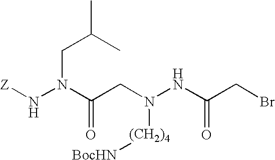

NMR ¹H (CDCl₃) δ 1.39 (d, 6H, J=6.3 Hz), 1.45 (s, 9H+4H), 1.88 (m, 1H), 2.81 (s, 2H), 3.55 (s, 2H), 3.76 (s, 2H), 4.73 (bs, 1H), 5.19 (s, 2H), 6.82 (bs, 1H), 7.39 (m, 5H), 8.41 (bs 1H). NMR ¹³C (CDCl₃) δ (ppm): 20.5 (q), 21.3 (t), 26.8 (t), 27.3 (d), 28.7 (q), 40.2 (t), 56.1 (t), 57.3 (t), 60.7 (t), 68.8 (t), 79.1 (s), 128.6 (d), 128.9 (d), 135.9 (d), 156.5 (s), 166.7 (s), 172.4 (s).

For the ketone groups: Trifluoroacetic anhydride (5.5 mmol, 1.1 equi) in solution in ether (5 ml) is added dropwise to a solution cooled down to 0° C., under stirring, of pseudodipeptoid (5 mmol, 1 equi) in ether (5 ml) and triethylamine (5.5 mmol, 1.1 equi). The reaction medium is left under stirring for 4 hours. When the triethylammonium salt precipitates, it is filtered and the filtrate is evaporated off under reduced pressure; otherwise, the medium is washed three times with 30 ml of water, the organic phase is dried over sodium sulphate and the solvent is evaporated off under reduced pressure. In both cases, the expected product precipitates slowly from ether in the cold state.

### F₃COC—N^{α}hLeu-N-azaLeu-Z: R¹=Z, R³═R⁴=i-Bu, R²═R⁵═H, R⁶═COCF₃. compound PR5

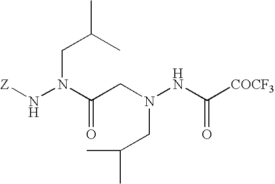

mp=105° C.; NMR ¹H (CDCl₃) δ 0.78 (d, 6H, J=6.6 Hz), 0.81 (d, 6H, J=6.6 Hz), 1.48 (m, 1H), 1.78 (m, 1H), 2.51 (broad d), 3.27 (broad d), 3.60 (s, 2H), 5.10 (s, 2H), 7.28 (m, 5H), 7.99 (broad s, 1H), 9.05 (broad s, 1H). NMR ¹³C (CDCl₃) δ (ppm) 20.4 (q), 20.8 (q), 26.7 (q), 26.8 (q), 56.0 (t), 59.2 (t), 65.8 (t), 68.4 (t), 113.4, 119.1, 128.9, 129.1, 135.7, 135.4, 172.2 (s).

For the borylated group: The borylated aldehyde (5.5 mmol, 1.1 equi) in solution in ether (10 mL) is added by small fractions to a solution under stirring of pseudopeptoid (5 mmol, 1 equi) in 5 ml of ether. A white precipitate forms instantaneously, but the medium is left under stirring for 1 hour. The white precipitate is filtered using sintered glass and is washed several times with ether.

### o-B(OH)₂-Ph-HC═N^{α}hLeu-N-azaLeu-Z: R¹=Z, R²═H, R³═R⁴=i-Bu, R⁵, R⁶═(HO)₂Bo-(C₆H₄)CH═. compound PR6

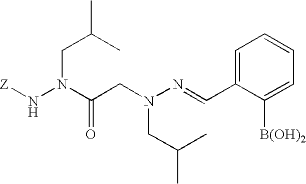

mp=128° C.; NMR ¹H(CDCl₃) δ (ppm) 0.91 (d, 6H), 1.12 (d, 6H), 1.86 (m, 1H), 2.15 (m, 1H), 3.16 (d, 2H), 3.78 (d, 2H), 4.53 (AB, 2H), 7.01 (t, 1H), 7.52(m, 8H), 8.21 (s, 2H), 9.30 (s, 1H), 11.27 (2H); NMR ¹³C (CDCl₃) δ (ppm) 20.2 (q), 21.0 (q), 26.3 (d), 27.2 (d), 55.5 (t), 57.0 (t), 60.1 (t), 68.2 (t), 127.7, 128.6, 128.9, 129.1, 131.2, 132.7, 136.1, 136.6, 138.3, 140.4, 155.8, 172.4 (s). Analysis calculated for C₂₅H₃₅N₄OBS: C, 62.25; H, 7.31; N, 11.61. Found: C, 62.20; H, 7.28; N, 11.71.

### o-B(OH)₂-Ph-HC═N^{α}hPhe-N-azaLeu-Z: R¹=Z, R²═H, R³=i-Bu, R⁴═CH₂Ph, R⁵, R⁶═(HO)₂Bo-(C₆H₄)CH═. compound PR7

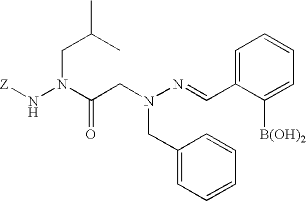

mp=135° C.; NMR ¹H (CDCl₃) δ (ppm) 1.01 (d, 6H), 1.99 (m, 1H), 3.43 (AB, 2H), 4.31 (AB, 2H), 4.74 (s, 2H), 5.31 (s, 2H), 7.33 (t, 1H), 7.52 (m, 14H), 7.61 (d, 1H), 8.14 (s, 2H), 10.1 (broad s, 1H); NMR ¹³C (CDCl₃) δ (ppm) 20.4 (q), 26.1 (t), 53.5 (t), 54.7 (t),57.8 (t), 67.1 (t), 125.8, 126.4, 127.5, 128.3, 128.4, 128.6, 128.7, 128.8, 132.6, 133.7, 136.5, 137.9, 139.9, 155.7 (s), 170.8 (s); Analysis calculated for C₂₈H₃₃N₄O₅B: C, 65.13; H, 6.44; N, 10.85; B, 2.09. Found: C, 64.79; H, 6.39; N, 10.68; B, 1.80.

### m-B(OH)₂-Ph-HC═N^{α}hPhe-N-azaLeu-Z: R¹=Z, R²═H, R³=i-Bu, R⁴═CH₂Ph, R⁵, R⁶═(HO)₂Bm-(C₆H₄)CH═. compound PR8

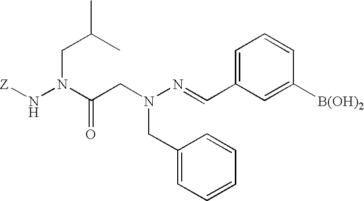

mp=157° C.; NMR ¹H (CDCl₃) δ (ppm) 0.77 (d, 6H), 1.76 (m, 1H), 3.17 (AB, 2H), 4.09 (AB, 2H), 4.54 (s, 2H), 5.08 (s,2H), 7.09 (m, 1H), 7.22 (m, 14H), 7.55 (d, 1H), 7.78 (s, 1H), 7.99 (s, 2H), 10.0 (broad s, 1H); NMR ¹³C (CDCl₃) δ (ppm) 20.3 (q), 26.1 (t), 54.5 (t), 54.6 (t), 58.3 (t), 67.1 (t), 127.0, 127.4, 127.9, 128.4, 128.6, 128.8, 131.1, 131.8, 135.9, 136.5, 138.2, 155.7 (s), 170.8 (s); Analysis calculated for C₂₈H₃₃N₄O₅B: C, 65.13; H, 6.44; N, 10.85; B, 2.09. Found: C, 65.39; H, 6.31; N, 11.23; B, 1.77.

### p-B(OH)₂-Ph-HC═N^{α}hPhe-N-azaLeu-Z: R¹=Z, R²═H, R³=i-Bu, R⁴═CH₂Ph, R⁵, R⁶═(HO)₂Bm-(C₆H₄)CH═. compound PR9

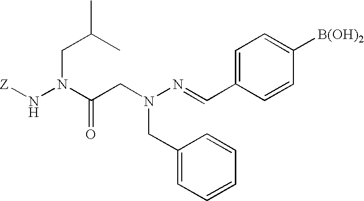

mp=205° C.; NMR ¹H (CDCl₃) δ (ppm) 0.86 (d, 6H), 1.85 (m, 1H), 2.61-2.99 (AB, 2H), 4.09-4.28 (AB, 2H), 4.65 (s, 2H), 5.17 (s,2H), 7.37 (m, 14H), 7.76 (m, 1H), 8.04 (s, 2H), 10.11 (broad s, 1H); NMR ¹³C (CDCl₃) δ (ppm) 20.3 (q), 26.2 (t), 53.1 (t), 54.6 (t), 58.5 (t), 67.1 (t), 124.4, 127.5, 128.1, 128.4, 128.8, 130.5, 134.7, 136.5, 138.1.138.5, 155.7 (s), 170.6 (s); Analysis calculated for C₂₈H₃₃N₄O₅B: C, 65.13; H, 6.44; N, 10.85; B, 2.09. Found: C, 64.47; H. 6.31; N, 10.82; B, 1.78.

### 2) Retro Hydrazinopeptoids

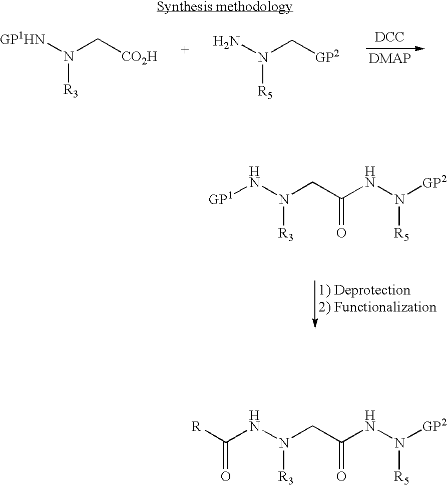

The monomer (A. Cheguillaume, I. Doubli-Bounoua, M. Baudy-Floc'h, P. Le Grel Synlett, 2000, 3, 331-334) deprotected at the N-terminal end (3.0 mmol), DMAP (0.1 mmol) and the monomer deprotected at the C-terminal end (3.0 mmol) are placed in solution in 50 mL of dichloromethane. The temperature of the reaction mixture is reduced to 0° C. and DCC (4.5 mmol, 1.5 equi.) is added by small fractions. After 5 minutes at this temperature, the reaction mixture is left under stirring overnight. The DCU formed is filtered on celite then the residue obtained is purified by flash chromatography. After washing with a 2N aqueous solution of hydrochloric acid, drying over sodium sulphate, the solvent is evaporated off. The dimer obtained is then deprotected at the N-terminal position and refunctionalized according to the methods above.

### BrH₂COC—N^{α}hLeu-N^{α}hLeu-Z: R¹=Z, R²=i-Bu, R³═R⁵═H, R⁶═COCH₂Br, R⁴=i-Bu. Compound PR1

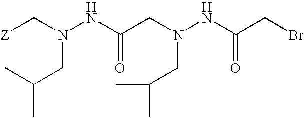

NMR ¹H (CDCl₃) δ 0.94 (2×d, 6H, J=7.6 Hz), 1.72 (m, 2×1H), 2.64 (d, 2H, 7 Hz), 2.73 (d, 2H, 7 Hz), 3.49 (s, 2H), 3.81 (s, 2H), 5.20 (s, 2H), 7.36 (m, 5H)), 8.11 (bs, 1H, 9.05 (bs, 1H).

### IV) Biological Analysis of the Compounds of Formula (Ib) and the Compounds P21 and P22

Proteasome is a protein structure involved in the processes of degradation of proteins regulating the cycle; it is a protein structure which possesses several proteolytic activities associated with different sub-units of the proteasome.

During the course of the cell cycle, dysfunctions of the proteasome can lead to anomalies in the development of the cycle which can be dramatic for the cell and the organism under consideration. As the proteasome inhibitors can stop cell cycle progression and cause apoptosis, they have become drugs which are potentially very useful for the treatment of certain tumours.

The proteasome inhibitors have a very serious anti-cancer potential and the numerous clinical studies currently in progress to assess their role as adjuvants in chemotherapy protocols, testify to this usefulness.

We have analyzed the effect of the retro compounds on mouse leukaemia cell cultures and confirm that among these products, three compounds have an antiproliferative effect. This effect has been observed on two other cell types, rat hepatocytes and a human breast cancer line. In these three systems, the proliferation index is close to zero, which indicates that under the effect of the compounds cited, the cells cease to grow.

The compounds PR7, PR6 and P21 (already described in the patent) are proliferation inhibitors and they do not affect the viability of the cells. The compounds PR1, PR2, PR3, PR5, PR8 and P22 (non-retro compound described above) inhibit proliferation and cause cell death with kinetics which vary from 2 to 12 hours according to the products.

These compounds are therefore particularly useful as anti-tumour molecules.

1. Use of compounds of the following general formula (I): 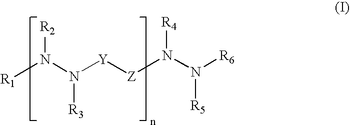
in which:
n represents an integer from 1 to 10, in particular n represents 1 or 2,
Y represents CH₂ and Z represents CO, or Y represents CO and Z represents CH₂,
R₁ and R₆, independently from one another, represent:
a hydrogen atom,
a group which can be used in the protection of the nitrogen atoms in peptide synthesis, such as the BOC, FMOC or Z group,
a group of formula —COR, or —CH₂COR in which R represents:
a hydrogen atom, except that, when R₁ is a hydrogen, this is presented in the form of a salt which is soluble in aqueous solvents, such as a trifluoroacetate salt,
an alkyl group with 1 to 10 carbon atoms, optionally substituted by one or more halogen atoms, such as the R groups representing —CF₃ or a —CH₂X group, X representing a halogen atom such as Cl or Br, or an abovementioned alkyl group substituted by a cyano group, such as the R group representing —CH₂—CN, or by a sulphurated group such as the R group representing —CH₂—SC₂H₅,
a —COORₐ group in which Rₐ represents H or an alkyl group, such as a methyl or ethyl group,
an —NH₂ primary amine or a secondary or tertiary amine group,
an alkoxy group, such as an —OMe methoxy, or —OEt ethoxy group,
a phenyl group,
a pyridinium group, such as the group of formula 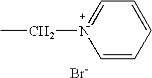
R₂, R₃, R₄ and R₅, independently from one another, representing:
a hydrogen atom,
an alkyl group with 1 to 10 carbon atoms, optionally substituted, in particular by one or more halogen atoms or by one or more amine or phenyl groups, such as the butyl, isobutyl, —(CH₂)₄NH₂, —CH₂Ph, —(CH₂)₄NHBoc groups,
or R₁ in combination with R₂, or R₆ in combination with R₅, represent a group of formula 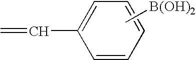
for the preparation of a medicament for the treatment of tumoral pathologies or neurodegenerative diseases such as Alzheimer's or Lehn's disease.

2. Use according to claim 1 of compounds of general formula (I) in which:
R₁ represents a BOC, FMOC, Z group or H, except that, when R₁ represents H, this is presented in the form of a salt, such as a trifluoroacetate salt of formula CF₃CO₂⁻, H₃N⁺—,
R₂ represents H or an alkyl group with 1 to 10 carbon atoms, such as an isobutyl group,
R₃ represents H or an alkyl group with 1 to 10 carbon atoms, such as an isobutyl group,
one of R₄ or R₅ represents H, whilst the other represents an alkyl group as defined above, and R₆ represents a group of formula —COR or —CH₂COR as defined above,
or R₅ in combination with R₆ represents a group of formula 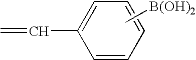
n represents 1 or 2,
Y and Z are as defined in claim 1.

3. Use according to claim 1 or 2, of compounds of general formula (I) in which R₅ represents H, and R₆ represents a —COR or —CH₂COR group in which R represents a —CH₂X group, X representing a halogen atom such as Cl or Br, or a pyridinium group.

4. Use according to one of claims 1 to 3, of compounds of general formula (I) in which R₅ represents H and R₆ represents a —COCH₂Br, —COCH₂Cl or 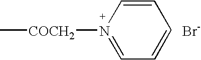
group.

5. Use according to one of claims 1 to 4, of compounds of general formula (I) in which R₁ and R₂ represent H.

6. Use according to one of claims 1 to 5, of compounds of general formula (I) in which Y represents CH₂ and Z represents CO, namely the compounds of the following formula (Ia): 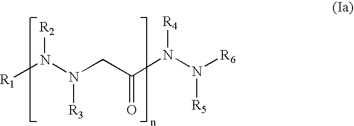
in which n, and R₁ to R₆ are as defined in one of claims 1 to 5.

7. Use according to one of claims 1 to 6, of the compounds of the following formulae: 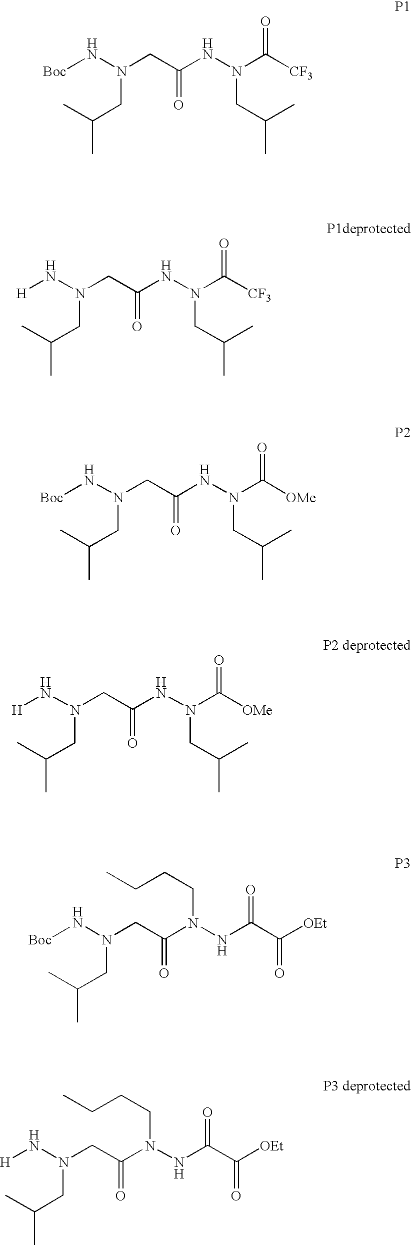 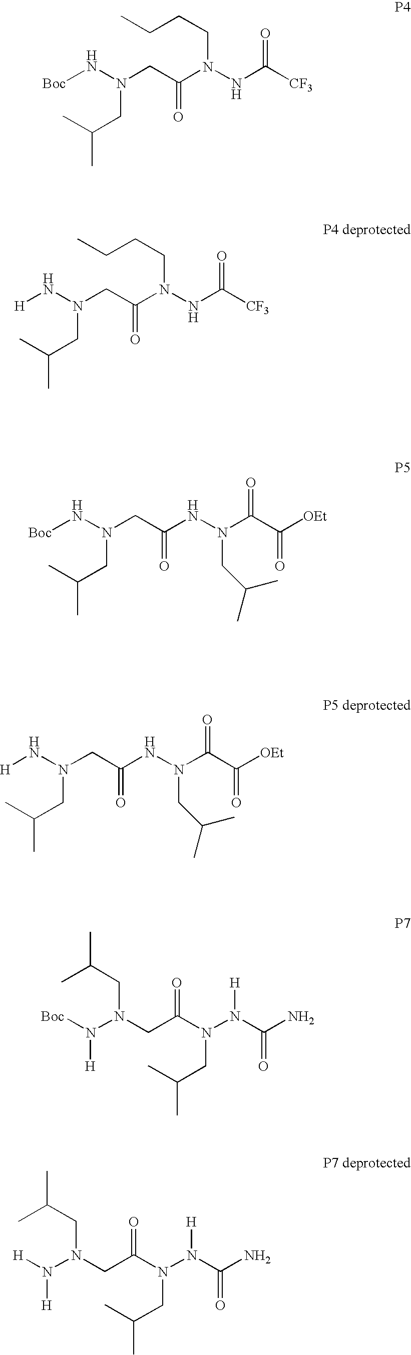 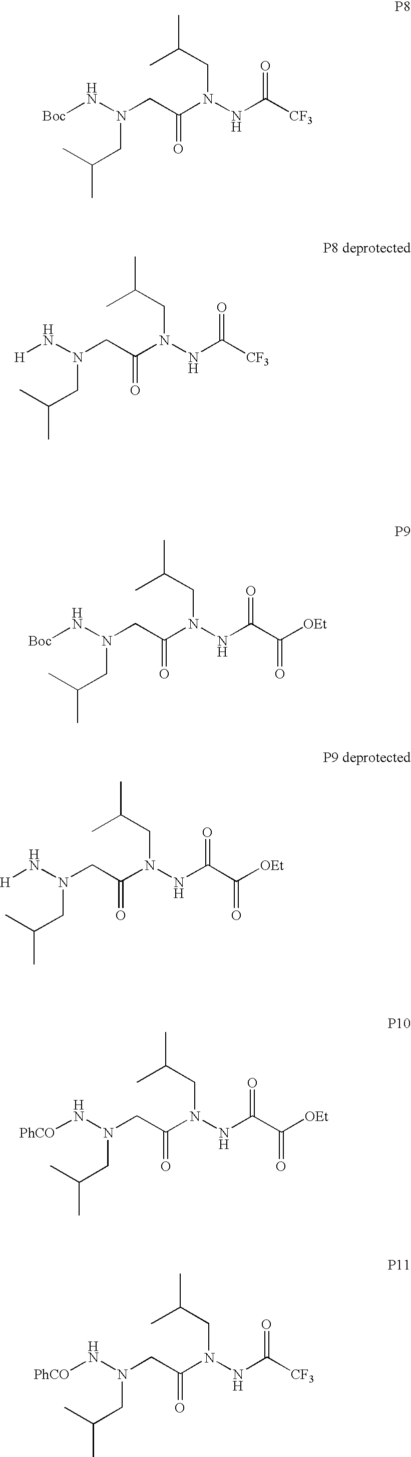 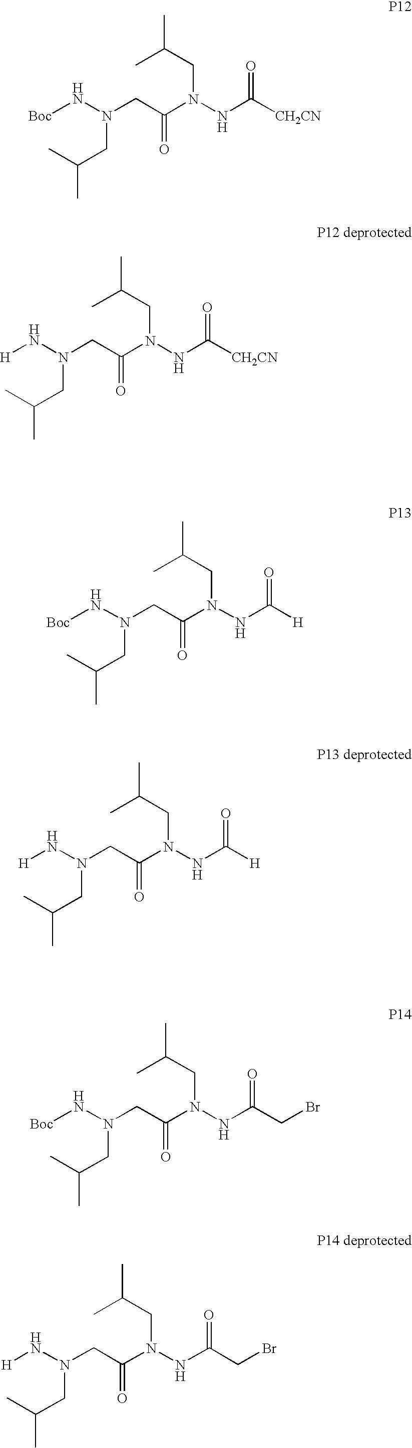 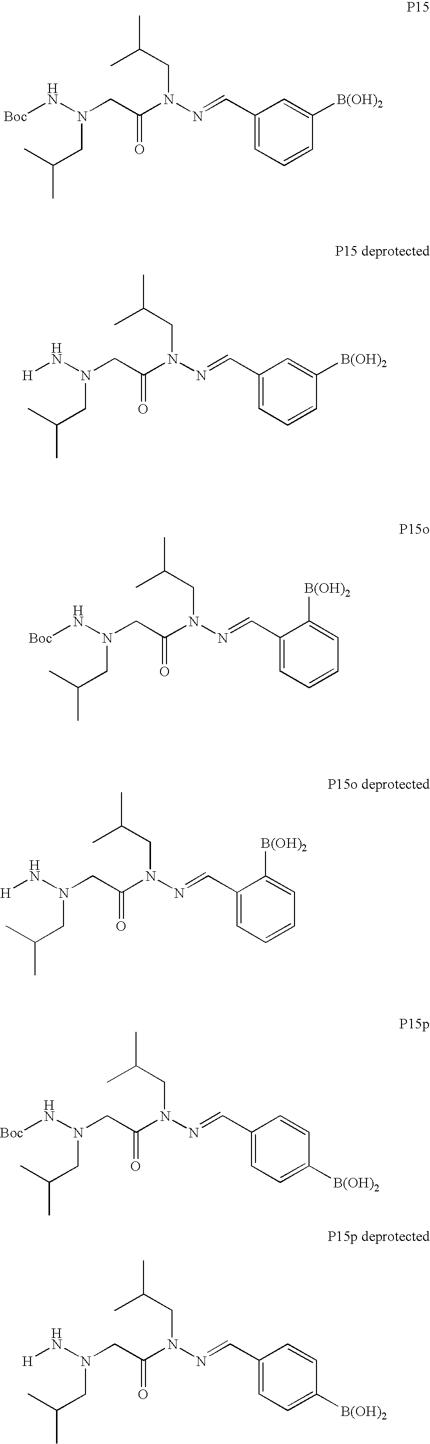 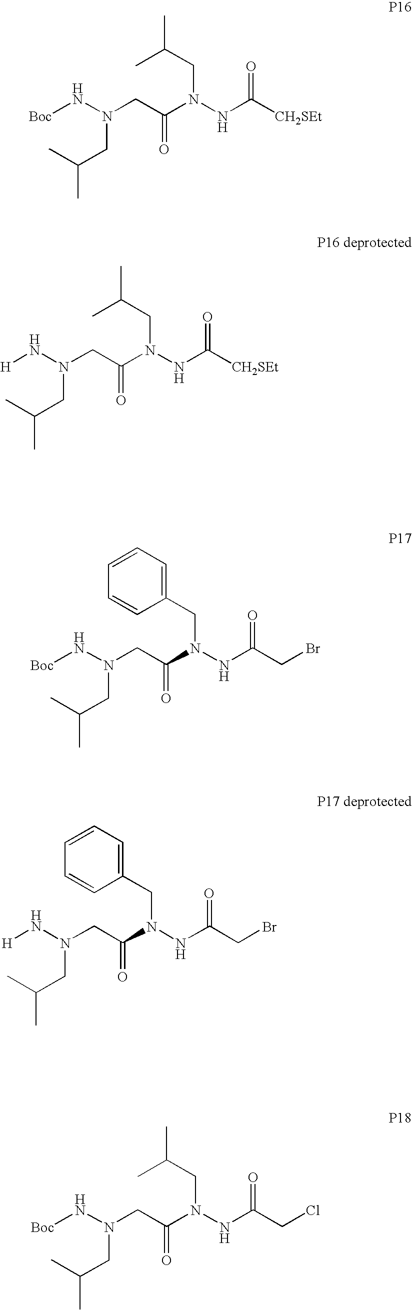 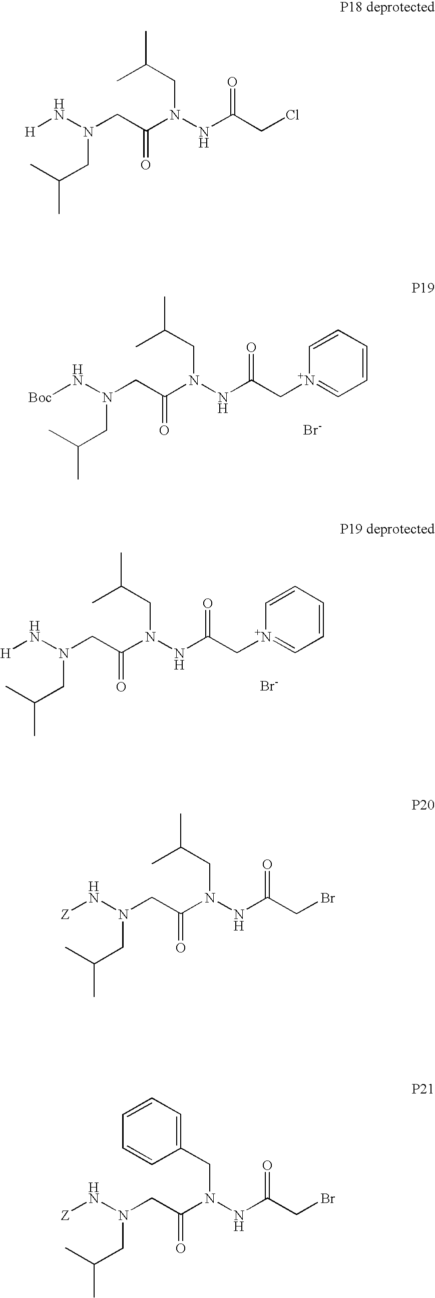 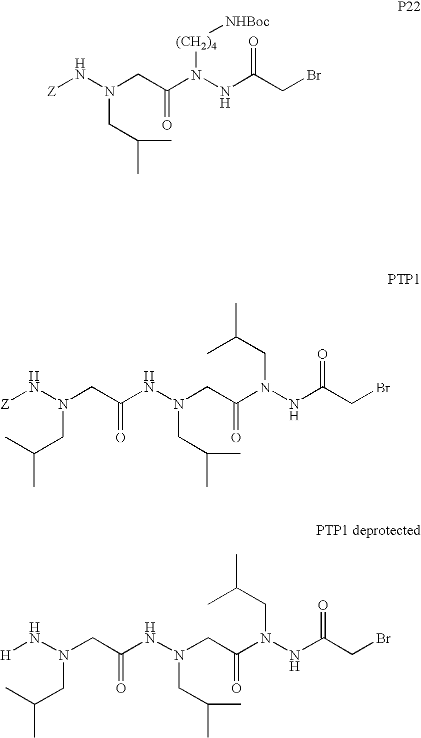
the deprotected compounds being in the form of a salt, such as a trifluoroacetate salt.

8. Use according to one of claims 1 to 7 of the compounds of the following formulae: 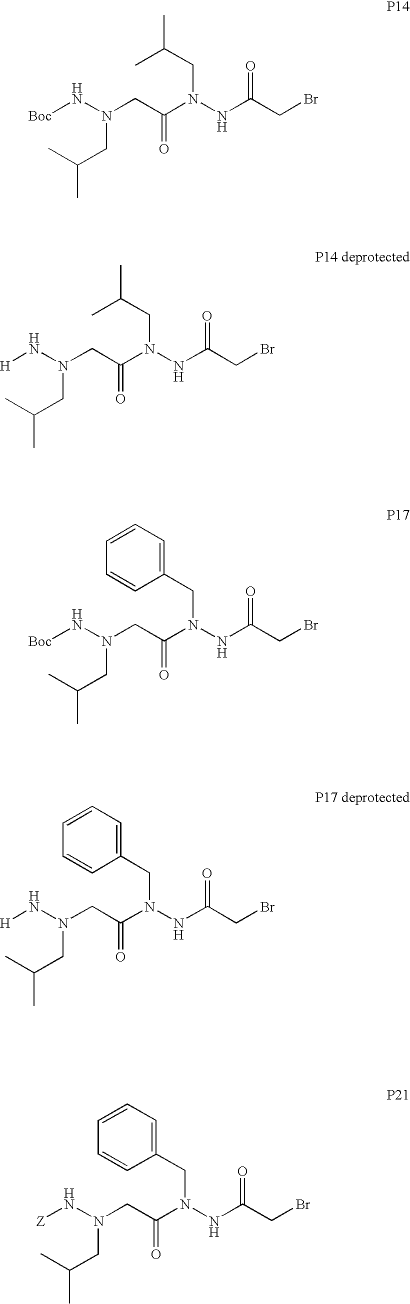 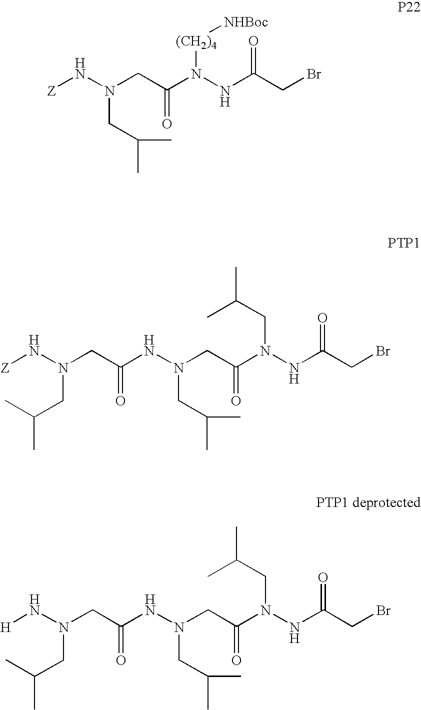
the deprotected compounds being in the form of a salt, such as a trifluoroacetate salt.

9. Use according to one of claims 1 to 8, of the compounds of general formula (I), in which Y represents CO and Z represents CH₂, namely the compounds of the following formula (Ib): 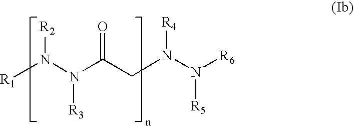
in which n, and R₁ to R₆ are as defined in claims 1 to 8.

10. Use according to compounds of formula (Ib) according to claim 10, in which:
n represents 1,
R₁ represents a Z group, or H, except that, when R₁ represents H, this is presented in the form of a salt, such as a trifluoroacetate salt of formula CF₃CO₂⁻, H₃N⁺—,
one of R₂ or R₃ represents H, whilst the other represents an alkyl group as defined above, in particular an isobutyl group,
R₄ represents an alkyl group as defined above, in particular an isobutyl group, or a —CH₂C₆H₅, or (CH₂)₄—NH₂, or —(CH₂)₄—NHBoc group,
R₅ represents H, and R₆ represents a group of formula —COR as defined above,
or R₅ in combination with R₆ represents a group of formula

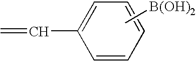

11. Use according to claim 10, characterized in that the compounds of formula (Ib) are those of the following formulae:

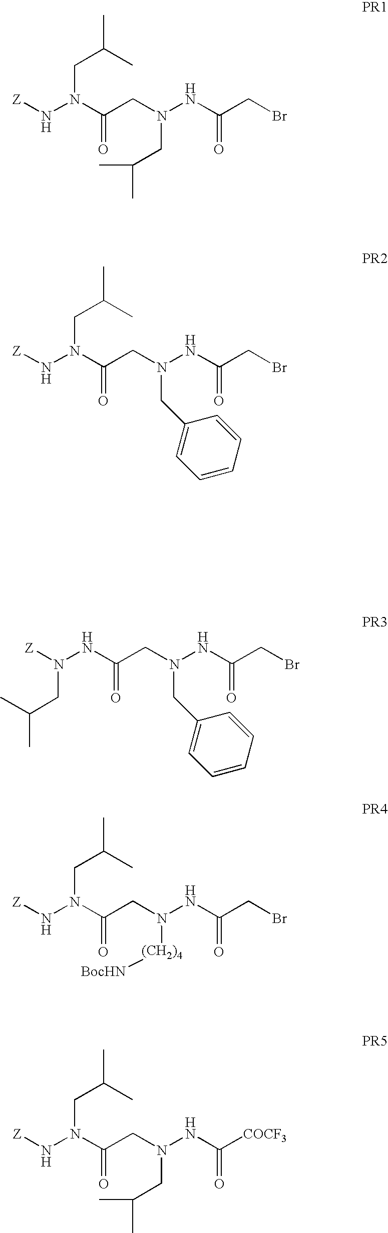

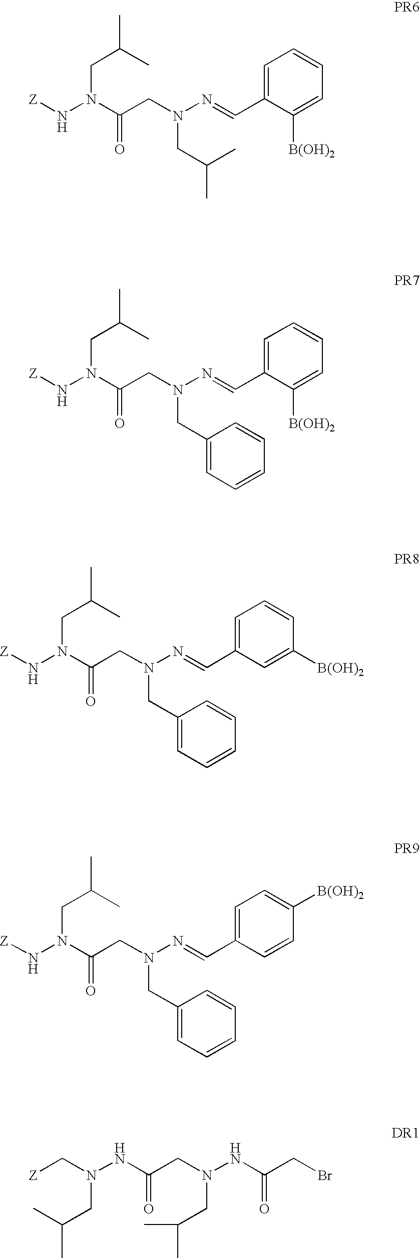

12. Use according to one of claims 1 to 11, for the preparation of a medicament intended for the treatment of cancers such as liver, colon, breast cancers, by inducing the entry into apoptosis of the cancerous cells by inhibition of the proteasome functions.

13. Compounds of general formula (Ia) in which:
n=1,
R₅ represents H, and R₆ represents a —COR or —CH₂COR group in which R represents a —CH₂X group, X representing a halogen atom such as Cl or Br, or a pyridinium group,
R₁ to R₄ are as defined in claims 1 to 5.

14. Compounds according to claim 13 of the following formulae: 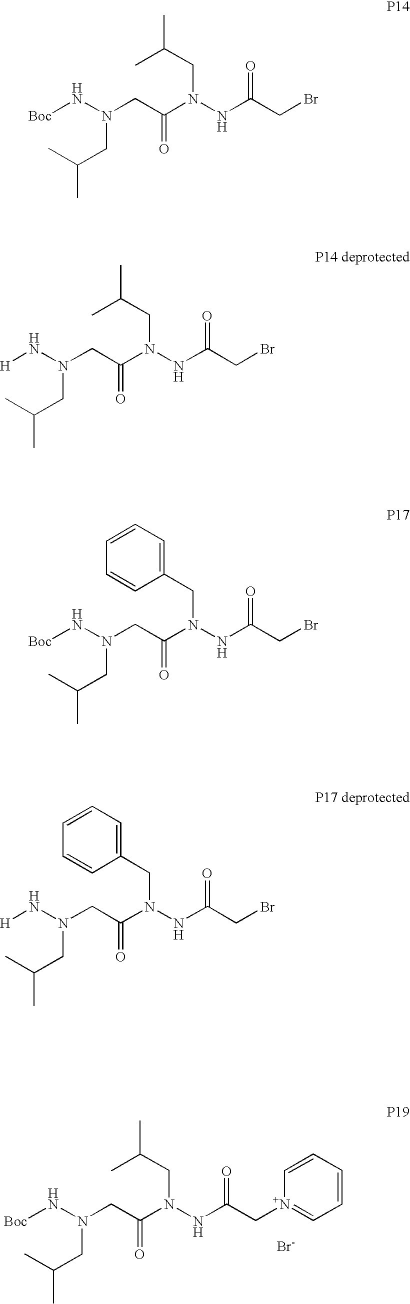 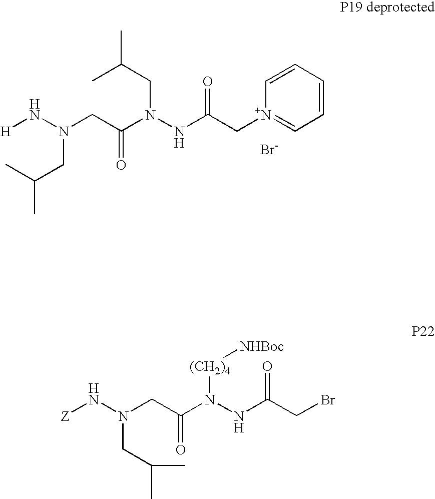
the deprotected compounds being in the form of a salt, such as a trifluoroacetate salt.

15. Compounds of general formula (Ia) in which:
n=2,
one of R₄ or R₅ represents H, whilst the other represents an alkyl group as defined in claim 1,
R₁, R₂, R₃ and R₆, are as defined in one of claims 1 to 5.

16. Compounds according to claim 15 of the following formulae: 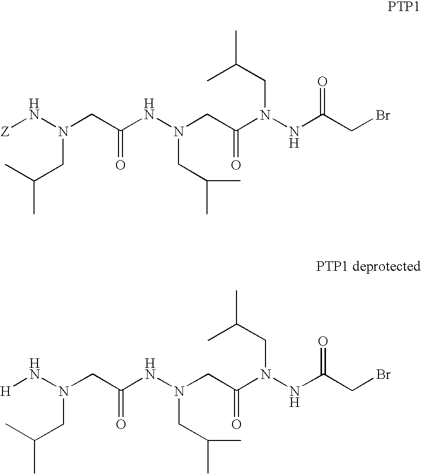
the deprotected compounds being in the form of a salt, such as a trifluoroacetate salt.

17. Compounds of the following general formula (Ib): 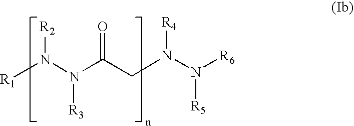
in which R₁ to R₆ are as defined in claim 1.

18. Compounds according to claim 17 of formula (Ib) in which:
n represents 1,
R₁ represents a Z group, or H, except that, when R₁ represents H, this is presented in the form of a salt, such as a trifluoroacetate salt of formula CF₃CO₂⁻, H₃N⁺—,
one of R₂ or R₃ represents H, whilst the other represents an alkyl group as defined above, in particular an isobutyl group,
R₄ represents an alkyl group as defined above, in particular an isobutyl group, or a —CH₂C₆H₅, or (CH₂)₄—NH₂, or —(CH₂)₄—NHBoc group,
R₅ represents H, and R₆ represents a group of formula —COR as defined above,
or R₅ in combination with R₆ represents a group of formula

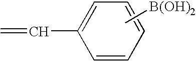

19. Compounds according to claim 17 or 18, of the following formulae:

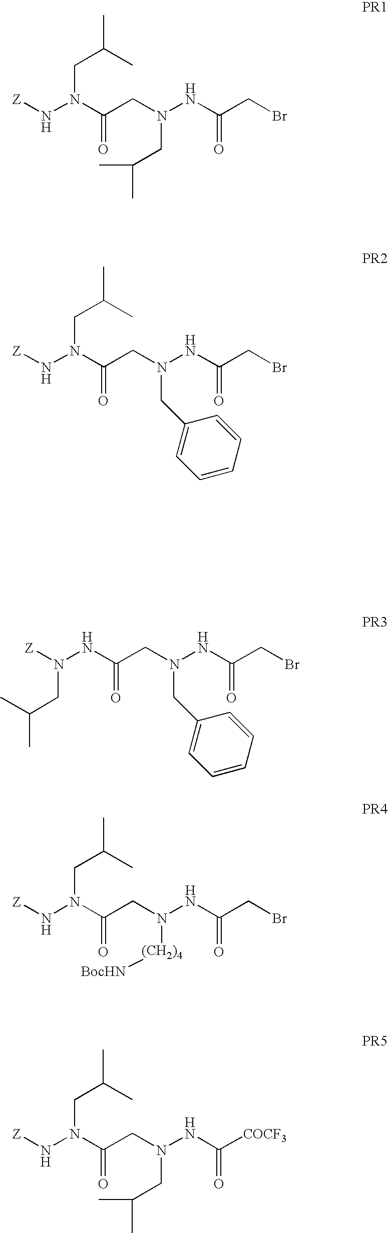

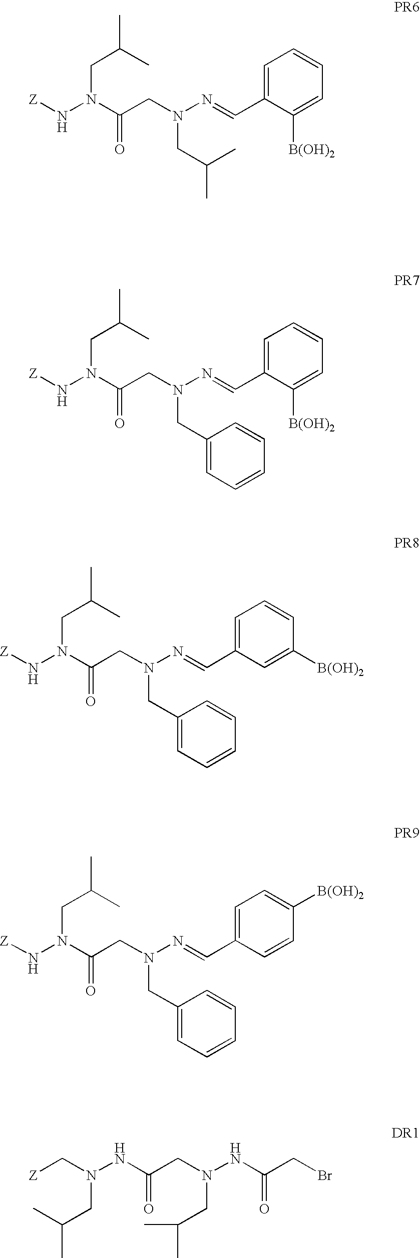

20. Pharmaceutical composition comprising a compound according to one of claims 10 to 19 in combination with a pharmaceutically acceptable vehicle.
